(19) United States
(12) Patent Application Publication
(10) Pub. No.: US 2005/0246784 A1
(43) Pub. Date: Nov. 3, 2005

(54) Method for identifying herbicidally active substances

(76) Inventors: Gunnar Plesch, Potsdam (DE); Astrid Blau, Stahnsdorf (DE); Klaus Daschner, Berlin (DE); Mathieu Klein, Berlin (DE)

Correspondence Address: NOVAK DRUCE DELUCA & QUIGG, LLP, 1300 EYE STREET NW, SUITE 400 EAST, WASHINGTON, DC, 20005, US

(21) Appl. No.: 10/467,962

(22) PCT Filed: Feb. 13, 2002
(86) PCT No.: PCT/EP02/01466

§ 371 Date: Aug. 14, 2003

(30) Foreign Application Priority Data
Feb. 16, 2001 (DE) 101078439
Mar. 23, 2001 (DE) 101255373

(51) Int. Cl. A01H 1/00; C12Q 1/68; A01N 25/00; C12N 15/82; C12N 5/04

(52) U.S. Cl. 800/278; 435/6; 435/419; 435/456; 504/116.1

(57) ABSTRACT

The present invention describes genes and methods for the isolation of genes which encode gene products which, if not present, lead to a greatly delayed growth and/or complete standstill of growth of Arabidopsis thaliana at the seedling level. The invention describes the use of these genes and gene products encoded by them for the discovery of novel herbicides. In addition, the present invention describes the use of the genes and gene products identified herein for the generation of plants which are resistant to herbicides. The present invention also relates to a method for identifying herbicidally active substances, to substances identified by this method and to their use as herbicides. The invention furthermore relates to nucleic acid constructs, vectors, organisms comprising the nucleic acid constructs and/or vectors, specifically transgenic plants comprising the nucleic acid constructs and/or vectors. Moreover, the invention relates to a method for identifying antagonists and to their use, antibodies and antisense RNA molecules and compositions comprising a herbicidal substance isolated by the methods according to the invention. Furthermore, the invention realtes to compositions comprising the antibody, the antisense nucleic acid and/or the antagonist.

Modern agriculture without the use of herbicides is inconceivable. The value of the herbicides used worldwide is currently estimated at approx. 30 billion DM. Even though a large number of highly effective and ecologically acceptable herbicides are currently available, the need for novel herbicides results firstly from the fact that weeds keep developing a resistance to currently employed herbicides, which means that some of these can no longer be employed, and secondly from the fact that some of the herbicides are ecologically disadvantageous. Herbicides will currently in many cases still be employed as mixtures which comprise several active ingredient components, which is of little benefit environmentally and which makes particularly high demands on the formulation.

Novel herbicides should be distinguished by as broad as possible a range of action, by ecological and toxicological acceptability and by low application rates.

The procedure so far for identifying and developing novel herbicides was characterized by applying potential active ingredients directly to suitable test plants. The disadvantage of this procedure is that relatively large amounts of substance are necessary to carry out the tests. This is rarely the case in the age of combinatorial chemistry, where a very large variety of substances can be prepared, albeit in small amounts. This is therefore an important limitation in development of novel herbicides. Also, the direct application to the plants to be tested means that even the first screening step makes extremely high demands on the substance, since not only the inhibition or other modulation of the activity of a cellular target (as a rule a protein or enzyme) is required, but the substance must initially reach this target in the first place, which means that even this first step makes demands on the test substance with regard to the uptake by the plant; permeability through the various cell walls and membranes, persistence for achieving the desired effect, and, finally, inhibition/modification of the activity of the desired target enzyme.

In view of these demands, it is therefore not surprising that, on the one hand, the identification of novel active ingredients causes increasingly high costs and, on the other hand, the number of active ingredients which are discovered decreases all the time.

It is an object of the present invention to provide targets for identifying novel herbicides and to provide novel herbicides and their use. We have found that this object is achieved by a method of identifying herbicidally active substances which comprises
a) the expression or the activity of the gene product of a nucleic acid or of a gene encompassing:
   aa) nucleic acid sequence with the sequence shown in SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, SEQ ID NO: 78, SEQ ID NO: 80, SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 98, SEQ ID NO: 100, SEQ ID NO: 102, SEQ ID NO: 106 or SEQ ID NO: 108 or
   bb) nucleic acid sequence which, owing to the degeneracy of the genetic code, can be derived by back translation from the amino acid sequences shown in SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 75, SEQ ID NO: 77, SEQ ID NO: 79, SEQ ID NO: 81, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 99, SEQ ID NO: 101, SEQ ID NO: 103, SEQ ID NO: 105, SEQ ID NO: 107 or SEQ ID NO: 109;
   cc) nucleic acid sequence which is a derivative or a fragment of the nucleic acid sequences shown in SEQ ID NO: 1, SEQ ID NO: 3, SEQ ID NO: 5, SEQ ID NO: 7 SEQ ID NO: 9, SEQ ID NO: 11, SEQ ID NO: 13, SEQ ID NO: 15, SEQ ID NO: 17, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, SEQ ID NO: 78, SEQ ID NO: 80, SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 98, SEQ ID NO: 100, SEQ ID NO: 102, SEQ ID NO: 106 or SEQ ID NO: 108 and which has at least 60% homology at the nucleic acid level;
   dd) nucleic acid sequence which encodes derivatives or fragments of the polypeptides with the amino acid sequences shown in SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 75, SEQ ID NO: 77, SEQ ID NO: 79, SEQ ID NO: 81, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 99, SEQ ID NO: 101, SEQ ID NO: 103, SEQ ID NO: 105, SEQ ID NO: 107 or SEQ ID NO: 109 which have at least 50% homology at the amino acid level;
   ee) nucleic acid sequence which encodes a fragment or an epitope of a polypeptide which specifically binds to an antibody, the antibody binding specifically to a polypeptide encoded by the sequence shown in SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, SEQ ID NO: 78, SEQ ID NO: 80, SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 98, SEQ ID NO: 100, SEQ ID NO: 102, SEQ ID NO: 106 or SEQ ID NO: 108;
   ff) nucleic acid sequence which encodes a fragment of a nucleic acid shown in aa) and which has m6A-methyltransferase activity, DNA-binding activity or DNA repair activity, for example as in the case of RAD 54, thioredoxin activity, VAV2 activity, fructokinase activity, zinc finger protein activity, LYTB activity, crepopin activity, leucine protein activity, DNAJ activity, CRS1 activity, alanyl-tRNA-synthetase activity, OEP86 activity, FMRF-amide-propeptide isolog activity, 26S proteosome subunit S5B activity, geranylgeranyl-pyrophosphate synthase activity, cecropin family signature, ftsH chloroplast protease activity, AIM1 activity, UDP-glucuronyl transferase activity, FPF1 activity, SHI-like zinc finger protein activity, Crp1 activity, CRS1 activity, translation releasing factor RF-1 activity, farnesyl transferase subunit A activity, ATP-dependent copper transporter RAN1 activity; and/or
   gg) nucleic acid sequence which encodes derivatives of the polypeptides with the amino acid sequences shown in SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 75, SEQ ID NO: 77, SEQ ID NO: 79, SEQ ID NO: 81, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 99, SEQ ID NO: 101, SEQ ID NO: 103, SEQ ID NO: 105, SEQ ID NO: 107 or SEQ ID NO: 109 which has at least 20% homology at the amino acid level and an equivalent biological activity; or
b) the expression or activity of an amino acid sequence which is encoded by a nucleic acid sequence of aa) to gg),
   and selecting the substances which reduce or block the expression or the activity.

Expression is understood as meaning the de-novo synthesis in vitro and in vivo of nucleic acids and of proteins encoded by nucleic acids, in particular that of the abovementioned nucleic acid and amino acid sequences. The term “expression” encompasses all of the biosynthetic steps which lead to the mature protein or to its degradation, for example transcription, translation, modification or processing of nucleic acids and proteins, for example pre- or post-transcriptional processing steps or post-translational modifications, for example splicing, editing, polyadenylation, capping, modifications of amino acids, for example glycosylation, methylation, acetylation, binding of coenzymes, phosphorylation, ubiquitation, binding of fatty acids, processing of signal peptides and the like.

For the purposes of the invention, transcription is to be understood as meaning RNA synthesis with the aid of an RNA polymerase in 5′→3′-direction using a DNA template. Translation is to be understood as meaning in-vitro and in-vivo protein biosynthesis. The term gene product is understood as meaning any molecule and any substance which originates owing to the expression, for example the transcription or translation, of a nucleic acid, for example of a DNA or RNA, for example of a gene, and the processing products which follow, such as, for example, the products obtained after splicing or modification. Thus, the term gene product is understood as meaning, for example, a processed RNA, for example a catalytic RNA, a functional RNA, such as tRNAs or rRNAs, or a coding RNA, such as mRNA. A protein, which is likewise understood as being a “gene product”, is synthesized as the consequence of an mRNA being translated. Proteins can be subjected to various processing steps as enumerated above by way of example during and after translation. Activity of the gene product is understood as meaning the biological activity or function of an RNA or of a protein, such as, for example, the enzymatic activity, the receptor binding property, the ability of binding certain proteins, nucleic acids or metabolites, for example in protein complexes, that is to say for example the regulatory property or the transporter function of the protein or of the RNA, as it occurs naturally in the organism. A reduced activity of the gene product is to be understood as meaning a reduction in the biological activity compared with the natural activity of the gene product by at least 10%, advantageously at least 20%, preferably at least 30%, especially preferably by at least 50% and very especially preferably by at least 70%. A blockage of the activity of the gene product means the complete, that is to say 100%, blockage of the activity or a part-blockage of the activity, preferably an at least 80%, especially preferably at least 90%, very especially preferably at least 95% blockage of the biological activity.

The activity of the gene product can also be reduced indirectly, for example by inhibiting the formation or activity of interaction partners, for example by influencing the metabolic cascade which the gene product is part of. For example, not only the enzyme in question, but also another enzyme or protein in the same metabolic cascade may be inhibited, and this leads to blocking of the subsequent, previous or any other enzyme involved and thus of the gene product described herein, for example by substrate or product inhibition. Such reductions by indirectly influencing the activity of an enzyme have been described in detail for example for the interaction of the glycolysis proteins and glycolysis metabolites and can be applied readily to other metabolic pathways in which the gene products described herein play a role. Likewise, the activity of a gene product used according to the invention can be reduced or inhibited by reducing or inhibiting the activity of interaction partners, for example other proteins in a a protein complex, using the gene product described herein. The result may be that all of the complex is no longer activated or formed in parts only, if at all, or is no longer subject to regulation. Examples of such ways of influencing the activity are described for example for spliceosomes, polymerases, ribosomes and the like.

A fragment is understood as meaning a part-sequence of a sequence described herein which encompasses fewer nucleotides or amino acids than the sequences described herein. For example, a fragment can encompass 1%, 5%, 10%, 30%, 50%, 70% or 90% of the original sequence. Preferably, a fragment encompasses 100, more preferably 50, even more preferably less than 20, amino acids of the nucleic acids in question.

The importance of the individual biosynthesis steps is known to the skilled worker and can be found for example in “Molecular Biology of the cell”, Alberts, New York, 1998, “Biochemie” Stryer, 1988, New York, “Biochemieatlas”, Michal, Heidelberg, 1999 or in “Dictionary of Biotechnology”, Coombs, 1992.

Thus, an embodiment relates to a method according to the invention wherein the expression or the activity of the abovementioned nucleic acids or amino acids is reduced or blocked by reducing or blocking the transcription, translation, processing and/or modification of at least one nucleic acid sequence or amino acid sequence according to the invention. The activity of one, two, three or more sequences can be reduced or blocked in accordance with the invention.

The method according to the invention can be carried out in individual separate approaches or, advantageously, in a high-throughput screening (HTS) and can be used for identifying herbicidally active substances or antagonists. Substances which interact with the abovementioned nucleic acids or their gene products can also be identified advantageously in the abovementioned method; these substances are potential herbicides whose action can be improved further by a traditional chemical synthesis.

Substances identified, or selected, by the method can be applied advantageously to a plant to test the herbicidal activity of the substances. Those substances which show a herbicidal activity are selected. In a further advantageous embodiment of the method, the substances can also be identified in an in-vitro test, in addition to the abovementioned in-vivo test method. Such an in-vitro test with the nucleic acids according to the invention or their gene products has the advantage that the substances can be screened rapidly and in a simple fashion for their biological action. Such tests are also advantageously suitable for what is known as HTS.

The method can be carried out with free nucleic acids such as DNA or RNA, free gene products or, advantageously, in an organism, the organism used being bacteria, yeasts, fungi or, advantageously, plants. The organisms used are, advantageously, the conditional or natural mutants of the sequences SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15 or SEQ ID NO: 17 SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, SEQ ID NO: 78, SEQ ID NO: 80, SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 9 8, SEQ ID NO: 100, SEQ ID NO: 102, SEQ ID NO: 106 or SEQ ID NO: 108. Conditional mutants are to be understood as being mutants which have to be induced first in order to show reduced expression, e.g. transcription or translation, of the abovementioned nucleic acids or the gene products encoded by them. An example of such conditional mutants are mutants in which the nucleic acids are located after a temperature-sensitive promoter which is nonfunctional at higher temperatures, that is to say which prevents transcription at higher temperatures, for example above 37° C. It is likewise possible to regulate the expression by means of an effector molecule, for example when the expression is controlled by means of a promoter capable of being regulated, such as the Tet systems.

A further embodiment according to the invention is a method of identifying an antagonist of proteins which are encoded by a nucleic acid sequence as is employed in the method according to the invention, in particular selected from the group consisting of:
a) a nucleic acid sequence with the sequence shown in SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, SEQ ID NO: 78, SEQ ID NO: 80, SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 98, SEQ ID NO: 100, SEQ ID NO: 102, SEQ ID NO: 106 or SEQ ID NO: 108 or
b) a nucleic acid which, owing to the degeneracy of the genetic code, can be derived by back translation from the amino acid sequences shown in SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 75, SEQ ID NO: 77, SEQ ID NO: 79, SEQ ID NO: 81, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 99, SEQ ID NO: 101, SEQ ID NO: 103, SEQ ID NO: 105, SEQ ID NO: 107 or SEQ ID NO: 109;
c) nucleic acid sequence which is a derivative or a fragment of the nucleic acid sequences shown in SEQ ID NO: 1, SEQ ID NO: 3, SEQ ID NO: 5, SEQ ID NO: 7 SEQ ID NO: 9, SEQ ID NO: 11, SEQ ID NO: 13, SEQ ID NO: 15, SEQ ID NO: 17, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, SEQ ID NO: 78, SEQ ID NO: 80, SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 98, SEQ ID NO: 100, SEQ ID NO: 102, SEQ ID NO: 106 or SEQ ID NO: 108 and which has at least 60% homology at the nucleic acid level;
d) nucleic acid sequence which encodes derivatives or fragments of the polypeptides with the amino acid sequences shown in SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 75, SEQ ID NO: 77, SEQ ID NO: 79, SEQ ID NO: 81, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 99, SEQ ID NO: 101, SEQ ID NO: 103, SEQ ID NO: 105, SEQ ID NO: 107 or SEQ ID NO: 109 which have at least 50% homology at the amino acid level;
e) nucleic acid sequence which encodes a fragment or an epitope of a polypeptide which specifically binds to an antibody, the antibody binding specifically to a polypeptide encoded by the sequence shown in SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, SEQ ID NO: 78, SEQ ID NO: 80, SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 98, SEQ ID NO: 100, SEQ ID NO: 102, SEQ ID NO: 106 or SEQ ID NO: 108;
f) nucleic acid sequence which encodes a fragment of a nucleic acid shown in aa) and which has m6A-methyltransferase activity, DNA-binding activity or DNA repair activity, for example as in the case of RAD 54, thioredoxin activity, VAV2 activity, fructokinase activity, zinc finger protein activity, LYTB activity, crepopin activity, leucine protein activity, DNAJ activity, CRS1 activity, alanyl-tRNA-synthetase activity, OEP86 activity, FMRF-amide-propeptide isolog activity, 26S proteosome subunit S5B activity, geranylgeranyl-pyrophosphate synthase activity, cecropin family signature, ftsH chloroplast protease activity, AIM1 activity, UDP-glucuronyl transferase activity, FPF1 activity, SHI-like zinc finger protein activity, Crp1 activity, CRS1 activity, translation releasing factor RF-1 activity, farnesyl transferase subunit A activity, ATP-dependent copper transporter RAN1 activity, syntaxin or syntaxin-like-protein activity, inositol-polyphosphate-5′-phosphatase activity, UDP—N-acetylmuramoyl-alanyl-D-glutamate-2,6-diaminopimelate ligase activity (murE), β-glucosidase activity, hydroxymethylglutaryl-CoA reductase activity, GDSL-motif-lipase/hydroxylase-like-protein activity, cellulose-synthase-like-protein activity, tRNA-glutamine synthetase activity, exonuclease-like-protein activity, sec-independent translocase protein TATC activity or selenium-binding-protein-like-protein activity; and/or
g) nucleic acid sequence which encodes derivatives of the polypeptides with the amino acid sequences shown in SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, SEQ ID NO: 78, SEQ ID NO: 80, SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 98, SEQ ID NO: 100, SEQ ID NO: 102, SEQ ID NO: 106 or SEQ ID NO: 108 which has at least 20% homology at the amino acid level or an equivalent biological activity; or
by following through the following process steps:
i) contacting cells which express the protein, or the protein, with a candidate substance;
ii) assaying the biological activity of the protein;
iii) comparing the biological activity of the protein with a standard activity in the absence of the candidate substance, a reduced biological activity of the protein indicating that the candidate substance is an antagonist.

“ii)” describes the testing of one of the above-described biological activities, e.g. an enzyme activity as stated in the examples, or a binding, preferably a strong binding between protein material and candidate material.

In an advantageous embodiment of the above-described method, the antagonist(s) identified under iii) is/are applied to a plant to test its/their herbicidal activity and the antagonist(s) which show(s) a herbicidal activity is/are selected.

The method according to the invention can be carried out in individual separate approaches in-vivo or in-vitro and/or advantageously jointly or, especially advantageously, in a high-throughput screening and can be used for identifying herbicidally active substances or antagonists.

The nucleic acid sequences identified or selected in the method according to the invention are essential for the growth and the development of higher plants. Suppression of the formation of the gene products, i.e. of expression, for example by exerting a specific effect on, for example, the transcription, the translation or the processing and/or of the suppression of the function or biological activity exerted by the encoded gene products by substances, advantageously low-molecular-weight substances with a molecular weight of less than 1000 daltons, advantageously less than 900 daltons, preferably less than 800 daltons, particularly preferably less than 700 daltons, very particularly preferably less than 600 daltons, advantageously with a Ki value of less than 1 mM and fewer than three hydroxyl groups on a carbon atom-containing ring or proteinogenic substances or a sense or antisense RNA or an antibody or antibody fragment in intact plants therefore leads to advantageously massive changes regarding the growth and the development of the plants in question. The substances identified in the method according to the invention are therefore suitable as herbicides in agriculture.

The nucleic acids SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 26, SEQ ID NO: 28, SEQ ID NO: 30, SEQ ID NO: 32, SEQ ID NO: 34, SEQ ID NO: 36, SEQ ID NO: 38 or SEQ ID NO: 40, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, SEQ ID NO: 78, SEQ ID NO: 80, SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 98, SEQ ID NO: 100, SEQ ID NO: 102, SEQ ID NO: 106 or SEQ ID NO: 108 which are used in the methods according to the invention are essential for organisms, preferably for plants. Some of the gene products of the sequences stated can be seen, for example, from the polypeptides of the sequences SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 75, SEQ ID NO: 77, SEQ ID NO: 79, SEQ ID NO: 81, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 99, SEQ ID NO: 101, SEQ ID NO: 103, SEQ ID NO: 105, SEQ ID NO: 107 or SEQ ID NO: 109. The genomic DNA of the sequences is represented in each case in SEQ ID NO: 19 (genomic DNA of line P95), SEQ ID NO: 20 (genomic DNA of line P9), SEQ ID NO: 21 (genomic DNA of line P38), SEQ ID NO: 22 (genomic DNA of line P44), SEQ ID NO: 23 (genomic DNA of line P77), SEQ ID NO: 24 (genomic DNA of line P102), SEQ ID NO: 43 (genomic DNA of line A 301034) and SEQ ID NO: 42 (genomic DNA of line A 300857). For lines A 300841 and A 300367, the genomic sequence is identical with SEQ ID NO: 32 and SEQ ID NO: 40, since no intron was found.

SEQ ID NO: 1 (=clone P95) encodes a protein (PID: g2460037), which has similarities with various m6A methyl transferases, among others from the mouse (NP_{—}062695) and humans (AAB71850).

SEQ ID NO: 3 (=clone P9) encodes a “DNA repair protein RAD54-like” protein homolog situated on chromosome 3 (EMBL/AP000419) of Arabidopsis. In the BLAST alignment, it has similarities with a series of DNA-binding proteins such as helicases, transactivators and the like (Emery et al., Gene 104 (1), 103-106, 1991). There exists a particularly great similarity with the Schizosaccharomyces pombe DNA repair protein RHP 54 (Muris et al., J. Cell. Sci. 109 (Pt 1), 73-81, 1996).

SEQ ID NO: 5 (=clone P38) encodes an ORF on chromosome 3 (EMBLNEW/AC023912), which encodes possibly a thioredoxin. The BLAST alignment reveals similarities with thioredoxins of various origins (Fraser et al., Nature 390 (6660), 580-586, 1997; Reith et al., Plant Mol. Biol. Rep., 13, 333-335, 1995).

Clone P44 (=SEQ ID NO: 7) encodes a protein of the TAC clone K15C23 (EMBLALERT/AB024024) without definitive homology in the BLAST search. Sequence alignments at the amino acid level show weak homologies with VAV2 proteins (small ras-like GTP-binding protein, Swissprot Q60992, Schuebel et al., Oncogene 13 (2), 363-371, 1996; Henske et al., Ann. Hum. Genet. 59 (Pt 1), 25-37, 1995).

SEQ ID NO: 9 (=clone P77) probably encodes a fructokinsase of the BAC clone F24B22 of chromosome 3 (EMBL/ATF24B22). BLAST alignments confirm this homology (swissprot P37829, Smith et al., Plant Physiol., 102 (3), 1043, 1993; Blatch et al., Gene, 95 (1), 17-23, 1990).

SEQ ID NO: 11 encodes a protein (Accession number BAB09578.1) which has weak homologies with various zinc finger proteins. It shows weak homologies with zinc finger proteins and DNA-binding proteins, such as, for example, the rat zinc finger protein 265 (Karginova, E. A., Am. J. Physiol. 273 (5 Pt 2), F731-F738 (1997)) for which a function for the regulation of transcription and/or splicing is assumed. In-vitro transcription or splicing assays have been described many times and are known to the skilled worker.

The protein encoded by SEQ ID NO: 13 (AB36712.1) has similarities with LYTB proteins, specifically with LYTB SYNY3 from Synechocystis (Q55643). The gene, or the protein encoded by the gene, is located on chromosome V (Accession number AB006706). Some ESTs (gb:Z34640, Z30476, AA605545, Z34228, H76883, Z26425) are known within the sequence of SEQ ID NO: 13.

SEQ ID NO: 15 encodes a hypothetical protein (CAB81447,1) which contains the crepropin family signature and shows weak homologies with myc-protooncogenes.

The protein encoded by SEQ ID NO: 17 (see also ESTAV528166) (AAF23295) has a certain homology with a leucine-rich human protein (Accession number P42704, Wang et al., In vitro Cell Dev. Biol. Amin. 1994, 30A(2): 111-114). It shows a substantial homology with a leucine-rich human protein (LRP130), whose function is unknown, but has weak homologies at the amino acid level with the consensus sequences of the ATP binding sites in ATP-dependent kinases and of the protein kinase C phosphorylation site of the epidermal growth factor receptor.

SEQ ID No: 26 encodes a protein which, over a region of 40 amino acids, has homologies with various DNAJ chaperone proteins (Heat Shock Protein 40), for example with DNAJ protein (Q9UXR9) from Methanosarcina thermophila (Hoffmann-Bang, Gene 238 (2), 387-395, 1999).

SEQ ID No: 28 encodes a hypothetical protein (CAC01859.1). The derived protein sequence shows marked homologies with the maize CRS1 gene product (AAG00595), which is required for splicing the group II intron of the chloroplast gene atpF.

SEQ ID No: 30 probably encodes an alanyl-tRNA syntethase (BAB10601.1).

SEQ ID No: 32 encodes a protein which has a strong homology with the chloroplast outer envelope 86 protein OEP86 from pea P. sativum, GenBank Accession Number Z31581 and has an ATP/GTP binding site motif (P-loop). ESTs (EST gb:AI998804.1, R90258, AA651438) are already known for this ORF (CAB80744.1).

SEQ ID No: 34 encodes a protein whose derived amino acid sequence (AAF25967.1) shows marked similarity with an Arabidopsis FMRF-amide propeptide isolog (gi|1871179).

SEQ ID No: 36 encodes an unknown protein (BAB02572.1) with weak homology with proteosomal protein 26S PROTEASOM SUBUNIT S5B, (Deveraux, 1995, J. Biol. Chem. 270 (40), 23726.

SEQ ID NO: 38 encodes an unknown protein.

SEQ ID NO: 40 encodes a protein with homology with a geranylgeranyl-pyrophosphate synthase (Bartley, Plant Physiol. 104, 1469-1470, 1994).

SEQ ID NO: 44 encodes a hypothetical protein of the ORF AT4g28590, which has a “cecropin” family signature (AA237-245).

SEQ ID NO: 46 encodes a putative ftsH chloroplast protease of the ORF At2g30950.

SEQ ID NO: 48 has similarity with the “AIM1” protein from Arabidopsis (CAB43915.1). Several ESTs, GB:Z31666, gb:Z33957, Z31666 have already been described for this ORF (F19B15.40). This protein is a peroxysomal tetrafunctional enzyme of the fatty acid metabolism.

SEQ ID NO: 50 encodes a UDP-glucuronyl-transferase-like protein of the ORF K21H1.19. It is highly likely that the transcription is modified or prevented, and the function of the gene thus destroyed, by the incorporation of the T-DNA into this position.

SEQ ID NO: 52 encodes a protein with unknown function of the ORF At2g15820.

SEQ ID NO: 54 encodes a protein of the ORF ATF12B17_{—}20, an FPF1-like (flowering promoting factor1) protein.

SEQ ID NO: 56 encodes a protein of the ORF ATF12B17_{—}10 with similarity with the Homo sapiens KIAA1038 protein.

SEQ ID NO: 58 encodes a protein of the ORF F24P17.10 with unknown function. The blastp alignment with standard settings reveals pronounced homologies with a nodulin/glutamate-ammonia ligase-like protein.

SEQ ID NO: 60 encodes an SHI-like zinc finger protein (short internodes) of the ORF K1L20.13.k

SEQ ID NO: 62 encodes a protein with similarity with crp1 from Zea mays, PIR:T01685 (ORF F4P12_{—}400). This ORF additionally contains the ESTs gb:AI999771.1, T45254, AA713158*.

SEQ ID NO: 64 encodes a putative protein with similarities with hypothetical proteins from Arabidopsis. The blastp analysis moreover reveals pronounced homology with CRS1 from Zea mays Accession AAG00595, which is a group II intron splicing factor (Till,B et al., RNA 7 (9), 1227-1238 (2001)) ORF (T21H19_{—}100).

SEQ ID NO: 66 encodes a protein of unknown function of the gene At5g24315.

SEQ ID NO: 68 encodes a protein ORF (T20O10_{—}10) with a high degree of similarity with translation releasing factor RF-1 from Synechocystis (PIR:S76914). The derived amino acid sequence comprises a prokaryotic type Class I peptide chain detachment factor motif, AA280-296.

SEQ ID NO: 70 encodes a protein with a high degree of similarity with an allergin (“minor allergen”) from Alternaria alternata (PIR2:S43111). The ESTS gb:R64949, AA651052 have already been found for ORF (C7A10.610).

SEQ ID NO: 72 encodes a protein with similarity with the alpha-subunit of a putative signal sequence receptor (ORF At2g1160).

SEQ ID NO: 74 encodes a protein with unknown function of the ORF AT4g01220, which contains the ESTs gb:AA597894, AA597304.

When subjected to blastp analysis with standard settings, SEQ ID NO: 76 reveals a similarity with oxidoreductases. The insertion of the T-DNA at this position interrupts the ORF F13O11.11. The cellular function of the encoded proteins is not known.

SEQ ID NO: 78 encodes the protein of the ORF F25L23_{—}240*, a farnesyl transferase subunit A.

SEQ ID NO: 80 encodes an ATP-dependent-copper-transporter-RAN1-like protein (ORF T19K24.18).

SEQ ID NO: 82 encodes the protein of the ORF F19B15.50 and has similarities with glycine-rich proteins. The ORF contains the ESTs gb:Z29181, T42831, Z34138, Z33797, Z30844.

SEQ ID NO: 84 encodes a protein with unknown function (ORF K21H1.18).

SEQ ID NO: 86 which, in the blastp alignment with standard setting reveals a high degree of homology with a variety of syntaxins and syntaxin-like proteins, including from plants. A plurality of ESTs (gb|F15498, gb|H37515, gb|T41906, gb|T22448, gb|W43356, gb|T20739) have already been identified for the ORF F3O9.4.

SEQ ID NO: 88 encodes the protein of the ORF AT2g31830, a putative inositol-polyphosphate-5′ phosphatase.

SEQ ID NO: 90 encodes the protein of the ORF F24D7.13, which encodes a putative UDP—N-acetylmuramoylalanyl-D-glutamate-2, 6-diaminopimelate ligase (murE).

SEQ ID NO: 92 encodes the protein of the ORF MRC8.5, which encodes a beta-glucosidase.

SEQ ID NO: 94 encodes the protein of the ORF F15M4.1, which encodes a hydroxymethylglutaryl-CoA reductase.

SEQ ID NO: 96 encodes the protein of the ORF MRN17.4. This ORF encodes a GDSL-motif-lipase/hydrolase-like protein.

SEQ ID NO: 98 encodes the protein of the ORF dl3705c, which encodes a cellulose-synthase-like protein.

SEQ ID NO: 100 encodes the protein of the ORF K5J14.11, which encodes a maize-crp1-protein-like protein.

SEQ ID NO: 102 encodes the protein of the ORF F4F7.26. This ORF encodes a putative t-RNA glutamine synthetase and shows homology in particular with the Lupinus luteus tRNA glutamine synthetase GI:2995454.

SEQ ID NO: 104 encodes the protein of the ORF MFB13.17, which encodes an exonuclease-like protein.

SEQ ID NO: 106 encodes the protein of the ORF At2g01110. This ORF encodes a putative “sec-independent” translocase protein TATC.

SEQ ID NO: 108 encodes the protein of the ORF F28J12.180, which encodes a putative protein. blastp analyses with standard setting reveal that the derived amino acid sequence not only has pronounced homogies with a variety of hypothetical and putative proteins, but also a high degree of similarity with the selenium-binding-protein-like proteins.

All of the abovementioned sequences were identified in Arabidopsis.

The suppression of the formation of the gene products or the suppression of the function or activity exerted by the encoded gene products in intact plants by a low-molecular-weight substance leads to reduced, preferably to suppressed growth; the development of the plant is drastically altered and suppressed.

Other clones which drastically alter the growth and the development of the plants are clones P95 and P99a, they are also advantageously suitable for identifying herbicides. The genomic region in which the insertion of the T-DNA was effected in clone P91 is located in a region for which no Open Reading Frame (=ORF) could have been predicted. In clone P91, the insertion of the T-DNA was effected in position 6632 of the P1 clone MUJ8 (AB028621) of the Arabidopsis chromosome III. The sequence can be found in AB028621. In the case of clone P99a, too, the insertion of the T-DNA (position 96710 of the BAC F10K1 [AC067971] of the Arabidopsis chromosome I) was effected at a location in the genome for which no ORF was predicted. The genomic sequence can be found in clone AC067971.

These abovementioned sequences or functional portions thereof make possible the identification of herbicides which can be used in agriculture, for example, via a method which comprises the following steps:
a) providing two lines of an organism which functionally express the gene products encoded by one of the sequences described for the method according to the invention, in particular SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, SEQ ID NO: 78, SEQ ID NO: 80, SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 98, SEQ ID NO: 100, SEQ ID NO: 102, SEQ ID NO: 106 or SEQ ID NO: 108 or by the above-described derivatives or fragments thereof which have the biological activity of these sequences, the expression level of the lines being different, for example by mutagenesis of one line and identification of a mutant with increased or reduced expression and/or activity of the abovementioned gene product in comparison with the starting line or, for example, by generating recombinant organisms, advantageously transgenic plants, plant tissues such as tissues of, for example, leaf, root, shoot or stem, plant seeds, plant calli or plant cells which functionally express the sequences described in accordance with the invention, in particular SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, SEQ ID NO: 78, SEQ ID NO: 80, SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 98, SEQ ID NO: 100, SEQ ID NO: 102, SEQ ID NO: 106 or SEQ ID NO: 108 or derivatives or fragments thereof which have the biological activity of these sequences;
b) addition of chemical compounds (which are to be tested for their herbidical activity) to the lines with the different expression or activity levels of the gene product, for example to recombinant organisms mentioned under a) and nonrecombinant starting organisms with a different, preferably lower, expression or activity level of the gene product;
c) determination of the biological activity, for example the enzymatic activity, the growth or the vitality of the two lines, for example of the recombinant organisms, in comparison with the nonrecombinant starting organisms after addition of chemical compounds in accordance with item b); and
d) selection of the chemical compounds which reduce or completely inhibit or block the biological activity, for example the enzymatic activity, the growth or the vitality of the line with the lower activity, for example which reduce or completely inhibit or block the biological activity, the growth or the vitality of the nonrecombinant organisms, of the chemical compounds determined in accordance with item c), in comparison with the treated recombinant organisms.

A herbicide which can be used in agriculture can also be identified when the recombinant organisms generated above in a) are tested in a method comprising the following steps:
(b) addition of chemical compounds to be tested for their herbicidal activity to the recombinant organisms mentioned under (a); and
(c) determination of the biological activity, for example of the enzymatic activity, the growth or the vitality of the recombinant organisms after addition of chemical compounds in accordance with (b) in comparison with the same untreated recombinant organisms; and
(d) selection of the chemical compound which reduces or completely inhibits or blocks the biological activity, for example the enzymatic activity, the growth or the vitality of the treated organisms in comparison with the untreated organisms.

Chemical compounds which reduce the biological activity, the growth or the vitality of the organisms are understood as meaning compounds which inhibit, i.e. reduce or block, the biological activity, the growth or the vitality of the organisms by at least 10%, advantageously by at least 30%, preferably by at least 50%, especially preferably by at least 70%, very especially preferably by at least 90%.

An advantageous substance is in particular a substance which damages the cell lines with low activity or, preferably, which is lethal but which does not damage, or is not lethal, for cell lines which have a higher activity of the gene product.

In general, lines of organisms can be employed in the abovementioned method which express the sequences according to the invention and in particular the gene products which are encoded by nucleic acids according to the invention, but which are not recombinant, as long as one line shows higher gene expression or activity of the gene product than another line. Such lines can occur naturally or be generated by mutageneses.

Assay systems which allow the identification of substances which suppress the formation of the gene products and/or the functions exerted by the gene products or the activity of the gene products in intact plants, plant parts, plant tissues or plant cells are known to the skilled worker. Examples which may be referred to here are test systems for the inhibition of enzymes such as the fructokinase activity as described by Tangney et al. (J. Mol. Microbiol. Biotechnol., 2 (1), 2000: 71-80), Martinez-Barajas et al. (Protein Expr. Purif., 1997, 11 (1), 41-46), Kanayama et al. (Plant Physiol., 1997, 113 (4), 1379-1384) or by Veiga-da-Cunha et al. (Protein Expr. Purif., 19 (1), 2000: 48-52). For example, such test systems can be used advantageously for what are known as inhibition assays for, for example, clone P77.

Further advantageous assay systems are, for example, fluorescence correlation spectroscopy (=FCS). With the aid of FCS (Brock et al., PNAS, 1999, 96, 10123-10128; Lamb et al., J. Phys. Org. Chem., 2000, 13654-658), it is possible to measure the diffusion of molecules over time, or to determine the difference of the bound versus free molecules. To this end, the molecules to be studied are fluorescence-labeled and, for example, a defined volume is placed into microtiter plates. The fluctuation of the molecules in the samples is driven by the Brownian motion. The translateral or rotational diffusion and conformation changes of the molecules can be monitored by a laser focussed into the sample and analyzed via a correlation. Owing to binding to other substances, the diffusion coefficient of the molecules changes. The binding of the molecules can be determined or quantified with the aid of various algorithms via the change in the diffusion coefficient. This method allows advantageous measurements to be carried out within a wide concentration range. The method is advantageously suitable for measuring recombinant proteins which are advantageously provided with what is known as a his-tag to facilitate purification via commercially available chromatography columns (Porath et al., Nature 1975, 258, 598-599). The protein purified in this way is finally provided with a fluorescence marker such as, for example, carboxytetramethylrhodamin or BODIPY® (for example, BODIPY 576/589 Angiotensin II, NEN® Life Science Products, Boston, Mass., USA). An excess of the compound or substance to be tested is subsequently added to the protein. The diffusion of the protein labeled in this way is finally determined using an FCS system (for example, ConfoCor2 with LSM 510, Carl Zeiss microscope, Jena, Germany).

A further advantageous detection method for the method according to the invention is what is known as the surface-enhanced laser desorption ionization method (=SELDI ProteinChip®). This method was first described by Hutchens and Yip (1980). Using this method, which was developed for the reproducible simultaneous identification of biomarkers or antigens (Hutchens and Yip, Rapid Commun. Mass Spectrom, 1993, 7, 576-580), the ligand-protein binding can be analyzed via mass spectrometry. Detection is via normal TOF detection (=time of flight). This method too allows recombinantly expressed proteins to be expressed and purified as described above. To carry out the measurement, the protein is immobilized on the SELDI ProteinChips®, for example via the his-tags which have already been used for purification or via ion interactions or hydrophobic interactions with the chip. The ligands are subsequently applied to the chip prepared in this way, for example using an autosampler. After one or more wash steps with buffers of various ionic strengths, the bound ligands are analyzed using the LDI laser. In doing this, the binding strength of the ligands is determined after each washing step.

A further advantageous detection method that may be mentioned is what is known as the Biacore method, where the refraction index at the surface upon binding of ligands and the protein bound to the surface is analyzed. In this method, a collection of small ligands is added sequentially to a measuring cell with the bound protein. The binding at the surface is determined by an increase in what is known as plasmon resonance (=SPR) by recording the laser refraction from the surface. In general, the change in refraction index which is determined for a change in the mass concentration at the surface, is equal for all proteins or polypeptides, that is to say this method can be used advantageously for a very wide range of proteins (Liedberg et al., Sens. Actuators, 1984, 4, 299-304). Again, as described above, recombinantly expressed proteins are used advantageously, and these proteins are bound to the Biacore chip (Uppsala, Sweden), for example via histidine residues (for example his-tag). The chip prepared in this way is again contacted with the ligands, for example with an autosampler, and the binding is measured via a detection system available from Biacore with the aid of the SPR signal, i.e. via the change in the refraction index.

The methods according to the invention have a series of advantages such as, for example:
novel potential targets for herbicidal active ingredients can be identified,
identification of herbicides which have as complete an action as possible, independently of the plant species,
substances which were generated by means of combinatorial chemistry and which can be distinguished by a great variety, but by low amounts which are available, can be tested efficiently for inhibitors of the newly identified targets
in the case of herbicides which, for example, have a very broad activity (nonselective herbicides or else selective herbicides), they permit resistance to these herbicides to be mediated to agriculturally useful plants (see description hereinbelow).

For example, substances which bind particularly specifically to, for example, a protein or protein fragment encoded by a nucleic acid whose expression is essential for the growth of the plants can be isolated using the abovementioned methods. This makes possible a simplified identification of possible inhibitors which inhibit proteins, for example in their enzyme properties, binding properties or other activities, for example also by inhibiting their processing, as described above, or which inhibit their transport within the cell or their import or export from organelles or cells. The substances identified in this way can also be applied to plants in a further step in screening methods as are known to the skilled worker and studied for their effect on the growth and the development. Thus, a selection is made from the infinite number of chemical compounds which would be suitable for a screening method, which selection makes it considerably easier for the skilled worker to identify herbicidal substances.

“Specific binding” is understood as meaning the specificity of interactions between two partners, for example proteins among themselves or between protein (enzyme) and substrate (substrate specificity). It is based on a specific molecular spatial structure. The destruction of this structure is termed denaturation, which is frequently irreversible, in most cases leading to loss of specificity. This biological activity depends greatly on the environmental conditions (buffer, temperature, contacts with nonphysiological surfaces like glass, or lack of cofactors). Enzyme-substrate or cofactor bindings, receptor-ligand bindings or antibody-antigen bindings are termed specific types of binding. In the simplest case, the enzyme-substrate interaction is described thermodynamically using the Michaelis-Menten equation. It describes the enzyme activity beyond what is known as the Michaelis-Menten constant, which, in turn, reflects the kinetics. This constant is also the unit of measurement for the enzyme activity which, in turn, reflects the specificity. Definition of the enzyme activity unit (in accordance with IUB): one unit U corresponds to the amount of enzyme which catalyzes the conversion of one micromole of substrate per minute under precisely defined experimental conditions. The specific activity is usually given in U/mg.

In a further step, the identified substances can then be applied to plants, microorganisms or cells, for example to plant cells, and the effect which they have on the metabolism of these plants can then be observed, for example enzyme activities, photosynthesis activities, metabolic activity, fixation rate, gas exchange, DNA synthesis, growth rates. These methods and many others which are known to the skilled worker are suitable for studying the viability of cells. Substances which reduce, in particular block, the growth of, for example cells, in particular plant cells, are then preferably suitable as a choice for herbicidal compositions.

Furthermore, studies into the application rates of the herbicides which have been found can be made at a very early stage. Moreover, the high specificity for, and efficacy against, weeds can be determined readily.

A multiplicity of chemicals can be tested rapidly and in a simple manner for herbicidal properties with the method according to the invention. The method allows a reproducible selection from a large number of substances of specifically those which are highly effective in order to subsequently carry out, on these substances, further in-depth tests which are familiar to the skilled worker.

The invention furthermore relates to a method of identifying inhibitors of plant proteins, which inhibitors have a potentially herbicidal action and which are encoded by the nucleic acid sequences used in the method according to the invention, by cloning the gene products, overexpressing them in a suitable expression cassette—for example in insect cells—breaking up the cells and employing the cell extract directly or after concentration or isolation of the protein in an assay system for measuring the biological activity in the presence of low-molecular-weight chemicals.

The invention therefore furthermore relates to substances identified by the methods according to the invention, the substance having a molecular weight of less than 1000 daltons, advantageously less than 900 daltons, preferably less than 800 daltons, especially preferably less than 700 daltons, very especially preferably less than 600 daltons, a Ki value preferably fewer than 1 mM and less than three hydroxyl groups on a carbon-atom-containing ring, or the substance being a proteinogenic substance, an antisense RNA, an inhibitory or an interfering RNA (RNAi).

The term “sense” refers to the strand of a double-stranded DNA which is homologous to the mRNA transcript. The “antisense” strand contains an inverted sequence which is complementary to that of the “sense” strand. For example, an antisense nucleic acid molecule comprises a nucleotide sequence which is complementary to the “sense” nucleic acid molecule which encodes a protein or an active RNA, for example complementary to the coding strand of a double-stranded cDNA molecule or complementary to an mRNA sequence. As a consequence, an antisense nucleic acid molecule can form hydrogen bonds with a sense nucleic acid molecule. The antisense nucleic acid molecule can be complementary to any of the coding strands shown here or only to part thereof. The term “coding region” refers to the region of a nucleic acid sequence whose codons are translated into amino acids. Also, the antisense nucleic acid molecule can be complementary to “noncoding regions” of the coding strand of the nucleic acid molecules shown. The term “noncoding region” refers to 5′- and 3′-sequences which flank the coding region and which are not translated into a polypeptide (for example also termed 5′- and 3′-untranslated regions). The nucleic acid molecule which encompasses an antisense sequence can also encompass further elements which are important for the expression and stability of the molecule, for example capping structures, poly-A-tails and the like.

The antisense nucleic acid molecule can be complementary to the entire coding region of an mRNA, but can also be an oligonucleotide which is complementary to only part of the coding or noncoding region of the mRNA. For example, an antisense oligonucleotide can be complementary to the region which encompasses or surrounds the translation start of the mRNA. For example, an antisense oligonucleotide can have a length of 10, 15, 20, 25, 30, 35, 40, 45 or 50 nucleotides. An antisense nucleic acid molecule can be generated by chemical synthesis and enzymatic ligation by methods known to the skilled worker. An antisense nucleic acid molecule can be synthesized chemically using naturally occurring nucleotides or nucleotides which have been modified in various ways, so that the biological stability of the molecules is increased or the physical stability of the duplex which forms between the antisense and sense nucleic acid is increased; for example, phosphorothioate derivatives and acridin-substituted nucleotides can be used. Examples of modified nucleotides which can be used for the generation of antisense nucleic acids encompass 5-fluorouracil, 5-bromouracil, 5-chlorouracil, 5-iodouracil, hypoxanthine, xanthine, 4-acetylcytosine, 5-(carboxyhydroxylmethyl)uracil, 5-carboxy-methylaminomethyl-2-thiouridine, 5-carboxymethylamino-methyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyaceticacidmethylester, uracil-5-oxyacetic acid (v), 5-methyl-2-thiouracil, 3-(3-amino-3-N-2-carboxypropyl)uracil, (acp3)w, and 2,6-diaminopurine.

As an alternative, antisense nucleic acid molecules can be prepared biologically using expression vectors into which polynucleotides with the opposite orientation have been cloned (so that RNA transcribed from the inserted polynucleotide is in antisense orientation relative to a target polynucleotide as has been described further above).

The antisense nucleic acid molecule can also be an “α-anomeric” nucleic acid molecule. An “α-anomeric” nucleic acid molecule forms specific double-strand hybrids with complementary RNAs in which the strands run in parallel with each other, in contrast to ordinary β units. The antisense nucleic acid molecule can encompass 2-0-methylribonucleotides or chimeric RNA-DNA-analogs.

Moreover, the antisense nucleic acid molecule can be a ribozyme. Ribozymes are catalytic RNA molecules with a ribonuclease activity which are capable of cleaving single-stranded nucleic acids, such as, for example, mRNA, to which they have a complementary region. Ribozymes (for example hammerhead ribozymes) can be used for catalytically or noncatalytically cleaving mRNA of the sequences described herein, thus preventing translation of the mRNA. A ribozyme which is specific for one of the nucleic acid sequences mentioned herein can be constructed on the basis of the cDNA sequences shown herein or on the basis of heterologous sequences which can be identified by the methods described herein. For example, a derivative of the Tetrahymena L-19 IVSRNA can be prepared by the nucleotide sequence of the active region being complementary to the nucleotide sequence which is cleaved in a coding mRNA. As an alternative, one of the coding or noncoding sequences described herein or of an mRNA thereof may also be used in order to select a catalytic RNA from an RNA pool (see, for example, Bartel, 1993, Science, 261, 1411). As an alternative, the expression can also be inhibited by nucleotide sequences which are complementary to a regulatory region of the nucleic acid sequences described herein (for example a promoter or enhancer) forming a triple-helical structure, which prevents transcription of the subsequent gene (for example Helene, 1991, Anticancer-Drug Des. 6, 596; Helene, 1992, Ann. NY Acad. Sci. 660, 27, or Maher, 1992, Bioassays, 14, 807).

“Antibodies” are understood as meaning, for example, polyclonal, monoclonal, human or humanized or recombinant antibodies or fragments thereof, single-chain antibodies or else synthetic antibodies. Antibodies according to the invention or fragments thereof are understood as meaning, in principle, all classes of immunoglobulins such as IgM, IgG, IgD, IgE, IgA or their subclasses such as the subclasses of IgG or their mixtures. Preferred are IgG and its subclasses such as, for example, IgG₁, IgG₂, IgG₂ₐ, IgG_{2b}, IgG₃ or IgG_{M}. Especially preferred are the IgG subtypes IgG₁ or IgG_{2b}. Fragments which may be mentioned are all truncated or modified antibody fragments with one or two binding sites which are complementary to the antigen, such as antibody portions with a binding site formed by light and heavy chain which corresponds to the antibody, such as Fv, Fab or F(ab′)₂ fragments or single-strand fragments. Preferred are truncated double-strand fragments such as Fv, Fab or F(ab′)₂. These fragments can be obtained, for example, via the enzymatic route by cleaving off the Fc portion of the antibodies using enzymes such as papain or pepsine, by chemical oxidation or by genetic manipulation of the antibody genes. Genetically engineered nontruncated fragments may also be used advantageously. The antibodies or fragments can be used alone or in mixtures. Antibodies can also be part of a fusion protein.

The substances identified can be chemically synthesized or microbiologically produced substances which may be found, for example, in cell extracts of, for example, plants, animals or microorganisms. Furthermore, while the substances mentioned may be known in the prior art, they may not be known as yet as herbicides. The reaction mixture can be a cell-free extract or encompass a cell or cell culture. Suitable methods are known to the skilled worker and are described generally, for example, in Alberts, Molecular Biology the cell, 3^{rd} Edition (1994), for example chapter 17. The substances mentioned may, for example, be added to the reaction mixture or the culture medium or injected into the cells or sprayed onto a plant.

Once a sample comprising an active substance identified by the method according to the invention has been identified, it is either possible to isolate the substance directly from the original sample, or the sample can be divided into different groups, for example when it is composed of a multiplicity of different components, in order to reduce the number of the different substances per sample and then to repeat the method according to the invention with such a “subsample” of the original sample. Depending on the complexity of the sample, the above-described steps can be repeated several times, preferably until the sample identified in accordance with the method according to the invention only encompasses a small number of substances or just one substance. Preferably, the substance identified in accordance with the method according to the invention, or derivatives of the substance, are formulated further so that it is suitable for use in plant breeding or in plant cell or tissue culture.

The substances which were tested and identified in accordance with the method according to the invention can be: expression libraries, for example cDNA expression libraries, peptides, proteins, nucleic acids, antibodies, small organic substances, hormones, PNAs or similar (Milner, Nature Medicin 1 (1995), 879-880; Hupp, Cell. 83 (1995), 237-245; Gibbs, Cell. 79 (1994), 193-198 and references cited therein). These substances can also be functional derivatives or analogs of the known inhibitors or activators. Methods for the preparation of chemical derivatives or analogs are known to the skilled worker. The abovementioned derivatives and analogs can be tested by prior-art methods. Moreover, computer-aided design or peptidomimetics can be used for preparing suitable derivatives and analogs. The cell or the tissue which can be used for the method according to the invention is preferably a host cell, plant cell or plant tissue according to the invention as described in the abovementioned embodiments.

Derivative(s) (the plural and the singular are to be taken as equivalent for the present application and its definitions) of the nucleic acids used in the methods according to the invention are, for example, functional homologs of the proteins encoded by SEQ ID NO: 1, SEQ ID NO: 3, SEQ ID NO: 5, SEQ ID NO: 7, SEQ ID NO: 9, SEQ ID No: 11, SEQ ID No: 13, SEQ ID No: 15, SEQ ID NO: 17, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, SEQ ID NO: 78, SEQ ID NO: 80, SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 98, SEQ ID NO: 100, SEQ ID NO: 102, SEQ ID NO: 106 or SEQ ID NO: 108 or their biological activity, that is to say proteins which carry out the same biological reactions as the proteins encoded by SEQ ID NO: 1, SEQ ID NO: 3, SEQ ID NO: 5, SEQ ID NO: 7, SEQ ID NO: 9, SEQ ID No: 11, SEQ ID No: 13, SEQ ID No: 15, SEQ ID NO: 17, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, SEQ ID NO: 78, SEQ ID NO: 80, SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 98, SEQ ID NO: 100, SEQ ID NO: 102, SEQ ID NO: 106 or SEQ ID NO: 108. These derivatives or genes are also suitable as herbicide targets.

The sequences described herein in accordance with the invention encode homologs with the proteins described in the examples and preferably have the activities specified for the homologs.

SEQ ID NO: 1 encodes a protein which has similarities with an m6A methyl transferase whose protein sequence is shown in SEQ ID NO: 2. SEQ ID NO: 3 encodes what is known as the “DNA-repair-protein-RAD-54-like-protein homolog”, whose protein sequence can be seen from SEQ ID NO: 4. SEQ ID NO: 5 can encode a thioredoxin, the protein sequence is represented in SEQ ID NO: 6. SEQ ID NO: 7 encodes an unknown protein whose sequence is shown in SEQ ID NO: 2. SEQ ID NO: 9 preferably encodes a fructokinase whose protein sequence can be seen from SEQ ID NO: 10. SEQ ID NO: 11 encodes a protein with weak homologies with various zinc finger proteins. The protein sequence can be seen from SEQ ID NO: 12. A protein which has similarities with LYTB proteins is encoded by SEQ ID NO: 13. SEQ ID NO: 14 indicates the protein sequence. Sequence SEQ ID NO: 15 encodes a protein with the sequence shown in SEQ ID NO: 16. This hypothetical protein encoded by SEQ ID NO: 15 contains what is known as a crepropin family signature and has weak homologies with myc protooncogenes. SEQ ID NO: 17 encodes a protein which has a certain homology with a leucine-rich human protein. The protein sequence can be seen from SEQ ID NO: 18. The nucleic acids SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, SEQ ID NO: 78, SEQ ID NO: 80, SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 98, SEQ ID NO: 100, SEQ ID NO: 102, SEQ ID NO: 106 or SEQ ID NO: 108 encode homologs or have similarity with proteins whose activity or function is shown above or in the examples. Each of the protein sequences is shown in SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 75, SEQ ID NO: 77, SEQ ID NO: 79, SEQ ID NO: 81, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 99, SEQ ID NO: 101, SEQ ID NO: 103, SEQ ID NO: 105, SEQ ID NO: 107 or SEQ ID NO: 109.

Derivatives are also understood as meaning those peptides which have at least 20%, or preferably 30%, more preferably 50%, even more preferably 70%, more preferably 80%, most preferably 90% or more homology with the polypeptides with the sequences shown in SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, SEQ ID NO: 78, SEQ ID NO: 80, SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 98, SEQ ID NO: 100, SEQ ID NO: 102, SEQ ID NO: 106 or SEQ ID NO: 108, and which have an equivalent biological activity in other organisms and can thus be regarded as functional homologs. This functional homology or equivalence can be demonstrated for example by the possible complementation of mutants in these functions.

The abovementioned nucleic acid sequence(s) or fragments thereof can be used advantageously for isolating further sequences such as, for example, genomic, cDNA or other sequences which are suitable as herbicide target, using homology screening.

The abovementioned derivatives can be isolated, for example, from other organisms, in particular eukaryotic organisms such as plants, such as, specifically, algae, mosses, dinoflagellates or fungi.

Derivatives or functional derivatives of the sequences stated in SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, SEQ ID NO: 78, SEQ ID NO: 80, SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 98, SEQ ID NO: 100, SEQ ID NO: 102, SEQ ID NO: 106 or SEQ ID NO: 108 are furthermore to be understood as meaning, for example, allelic variants which have at least 60% homology, advantageously at least 70% homology, preferably at least 80% homology, especially preferably at least 85% homology, very especially preferably 90% homology at the derived amino acid level. The homology was calculated over the entire amino acid region. The programs PileUp, BESTFIT, GAP, TRANSLATE or BACKTRANSLATE (=part of the UWGCG package, Wisconsin Package, Version 10.0-UNIX, January 1999, Genetics Computer Group, Inc., Deverux et al., Nucleic. Acid Res., 12, 1984: 387-395) were used (J. Mol. Evolution., 25, 351-360, 1987, Higgins et al., CABIOS, 5 1989: 151-153). The amino acid sequences derived from the abovementioned nucleic acids can be seen from SEQ ID NO: 2, SEQ ID NO: 4, SEQ ID NO: 6, SEQ ID NO: 8, SEQ ID NO: 12, SEQ ID NO: 14, SEQ ID NO: 16, SEQ ID NO: 18, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 75, SEQ ID NO: 77, SEQ ID NO: 79, SEQ ID NO: 81, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 99, SEQ ID NO: 101, SEQ ID NO: 103, SEQ ID NO: 105, SEQ ID NO: 107 or SEQ ID NO: 109. Homology is to be understood as meaning identity, that is to say that the amino acid sequences have at least 40, 50, 60 or 70%, more preferably 80%, even more preferably 90%, most preferably 95% or more identity. The sequences according to the invention have at least 45 or 55%, preferably at least 60 or 65%, especially preferably 75%, very especially preferably at least 80%, even more preferably 90 or 95% or more homology at the nucleic acid level.

The term derivatives and the term “fragments” furthermore also encompass subregions or fragments of the abovementioned sequences or their homologous sequences of at least 50 amino acids, advantageously of at least 40 amino acids, preferably of at least 30 amino acids, especially preferably of at least 20 amino acids, very especially preferably of at least 10 amino acids, which make it possible selectively to identify interacting substances. The term “fragment”, “sequence fragment” or “part-sequence” denotes a truncated sequence of the original sequence. The truncated sequence (nucleic acid or protein) can have different lengths, the minimum sequence length being a sequence length which has at least one comparable function, for example binding properties, or activity of the original sequence. Such methods are, for example, SELDI, FCS or Biocore as described above, which are known to the skilled worker.

Equally encompassed are thus nucleic acids which encode a fragment or an epitope of a polypeptide which specifically binds to an antibody which specifically binds to a polypeptide described in accordance with the invention, in particular which is encoded by one of the sequences shown in SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, SEQ ID NO: 78, SEQ ID NO: 80, SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 98, SEQ ID NO: 100, SEQ ID NO: 102, SEQ ID NO: 106 oder SEQ ID NO: 108. Fragments or epitopes of a polypeptide which specifically interact with such an antibody have a significant homology with regard to the spatial structure to the polypeptides described herein, at least in subregions. Preferably, they also have high homology at the amino acid level with the abovementioned sequences, preferably 20%, with 40% being more preferred, 60% more preferred, 80% even more preferred, but 90% or more being most preferred. The spatial structure of a polypeptide, however, is essentially one of the factors responsible for the interactions of the polypeptide with other compounds and, if appropriate, for its enzymatic activity. Accordingly, fragments may be employed in the processes according to the invention whose sequence has only a low degree of homology with the above-described polypeptides, but whose spatial structure has a high degree of homology with the above-described polypeptides, that is to say those comprising epitopes of the sequences described herein, in order to find interactants which then inhibit or inactivate the polypeptides described herein. Fragments which encompass epitopes of the polypeptides according to the invention can also be used to “occupy” the interactants of the polypeptides according to the invention, i.e. to prevent their interaction with the polypeptides according to the invention. To this end, it is advantageous for the fragments to have a greater affinity to a binding partner than the naturally occurring polypeptide. Likewise encompassed are fragments which are encoded by nucleic acids according to the invention and which encompass one of the abovementioned biological activities.

Allelic variants encompass in particular functional variants which can be obtained from the sequence shown in SEQ ID NO: 1, SEQ ID NO: 3, SEQ ID NO: 5; SEQ ID NO: 7, SEQ ID NO: 9, SEQ ID NO: 11, SEQ ID NO: 13, SEQ ID NO: 15, SEQ ID NO: 17, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, SEQ ID NO: 78, SEQ ID NO: 80, SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 98, SEQ ID NO: 100, SEQ ID NO: 102, SEQ ID NO: 106 or SEQ ID NO: 108 by deletion, insertion or substitution of nucleotides, the biological, e.g. enzymatic activity or binding properties of the derived proteins which are synthesized being retained.

Starting from, for example, the DNA sequences described in SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 26, SEQ ID NO: 27, SEQ ID NO: 30, SEQ ID NO: 32, SEQ ID NO:.34, SEQ ID NO: 36, SEQ ID NO: 38, SEQ ID NO: 40, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, SEQ ID NO: 78, SEQ ID NO: 80, SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 98, SEQ ID NO: 100, SEQ ID NO: 102, SEQ ID NO: 106 or SEQ ID NO: 108 or portions of these sequences, such DNA sequences can be isolated from other eukaryotic organisms such as, for example, microorganisms such as yeasts, fungi, ciliates, plants such as algae, mosses or other plants, with the aid of the nucleic acid sequences according to the invention, for example using customary hybridization methods or PCR technology. These DNA sequences hybridize with the abovementioned sequences under standard conditions. For hybridization, it is advantageous to use short oligonucleotides, for example of the conserved or other regions, which can be determined via alignment with other related genes in the manner known to the skilled worker. However, longer fragments of the nucleic acids according to the invention or the complete sequences may also be used for hybridization. These standard conditions vary depending on the nucleic acid used: oligonucleotide, longer fragment or complete sequence, or on the type of nucleic acid, DNA or RNA, which is used for the hybridization. Thus, for example, the melting points for DNA:DNA hybrids are approximately 10° C. lower than those of DNA:RNA hybrids of the same length.

Standard conditions are to be understood as meaning, for example, temperatures between 42 and 58° C. in an aqueous buffer solution with a concentration of between 0.1 to 5×SSC (1×SSC=0.15 M NaCl, 15 mM sodium citrate, pH 7.2) or additionally in the presence of 50% formamide such as, for example, 42° C. in 5×SSC, 50% formamide, depending on the nucleic acid. The hybridization conditions for DNA:DNA hybrids are advantageously 0.1×SSC and temperatures of between approximately 20° C. and 45° C., preferably between approximately 30° C. and 45° C. For DNA:RNA hybrids, the hybridization conditions are advantageously 0.1×SSC and temperatures of between approximately 30° C. and 55° C., preferably between approximately 45° C. and 55° C. These temperatures stated for the hybridization are examples of calculated melting point values for a nucleic acid with a length of approximately 100 nucleotides and a G+C content of 50% in the absence of formamide. The experimental conditions for DNA hybridization are described in specialist textbooks of genetics such as, for example, Sambrook et al., “Molecular Cloning”, Cold Spring Harbor Laboratory, 1989, and can be calculated by formulae known to the skilled worker, for example as a function of the length of the nucleic acids, the type of the hybrids or the G+C content. The skilled worker will find further information on hybridization in the following textbooks: Ausubel et al. (eds), 1985, Current Protocols in Molecular Biology, John Wiley & Sons, New York; Hames and Higgins (eds), 1985, Nucleic Acids Hybridization: A Practical Approach, IRL Press at Oxford University Press, Oxford; Brown (ed), 1991, Essential Molecular Biology: A Practical Approach, IRL Press at Oxford University Press, Oxford.

Derivatives are furthermore to be understood as meaning homologs of the sequence SEQ ID No: 1, SEQ ID NO: 3, SEQ ID No: 5, SEQ ID No: 7, SEQ ID NO: 9, SEQ ID NO: 11, SEQ ID NO: 13, SEQ ID NO: 15, SEQ ID NO: 17, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, SEQ ID NO: 78, SEQ ID NO: 80, SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 98, SEQ ID NO: 100, SEQ ID NO: 102, SEQ ID NO: 106 or SEQ ID NO: 108 for example eukaryotic homologs, truncated sequences, simplex DNA of the coding and noncoding DNA sequence or RNA of the coding and noncoding DNA sequence.

Homologs of the sequences SEQ ID NO: 1, SEQ ID NO: 3, SEQ ID No: 5, SEQ ID No: 7, SEQ ID NO: 9, SEQ ID NO: 11, SEQ ID NO: 13, SEQ ID NO: 15, SEQ ID NO: 17, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, SEQ ID NO: 78, SEQ ID NO: 80, SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 98, SEQ ID NO: 100, SEQ ID NO: 102, SEQ ID NO: 106 or SEQ ID NO: 108 are furthermore understood as meaning derivatives such as, for example, variants from other organisms, for example other plants. These variants can be modified by one or more nucleotide substitutions, by insertion(s) and/or deletion(s) without, however, adversely affecting the functionality or biological activity of the variants. They preferably have a homology of at least 20% and an equivalent biological activity.

The nucleic acids SEQ ID NO: 1, SEQ ID NO: 3, SEQ ID No: 5, SEQ ID No: 7, SEQ ID NO: 9, SEQ ID NO: 11, SEQ ID NO: 13, SEQ ID NO: 15, SEQ ID NO: 17, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, SEQ ID NO: 78, SEQ ID NO: 80, SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 98, SEQ ID NO: 100, SEQ ID NO: 102, SEQ ID NO: 106 oder SEQ ID NO: 108, and their fragments and derivatives which are used in the method according to the invention are therefore advantageously suitable for isolating further essential, novel genes from other organisms, preferably plants.

The nucleic acid sequences according to the invention SEQ ID NO: 1, SEQ ID NO: 3, SEQ ID No: 5, SEQ ID No: 7, SEQ ID NO: 9, SEQ ID NO: 11, SEQ ID NO: 13, SEQ ID NO: 15, SEQ ID NO: 17, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, SEQ ID NO: 78, SEQ ID NO: 80, SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 98, SEQ ID NO: 100, SEQ ID NO: 102, SEQ ID NO: 106 or SEQ ID NO: 108 whose gene products which are encoded by them are used in the method according to the invention can be of synthetic or natural origin or comprise a mixture of synthetic and natural DNA components, or else be composed of various heterologous gene segments of different organisms. In general, synthetic nucleotide sequences are prepared which have codons which are preferred by the host organisms in question, for example plants. As a rule, this leads to optimal expression of the heterologous genes. These codons which are preferred by plants can be determined from codons with the highest protein frequency which are expressed in most of the plant species of interest. An example of Corynebacterium glutamicum is provided in: Wada et al. (1992) Nucleic Acids Res. 20:2111-2118). Such experiments can be carried out with the aid of standard methods and are known to those skilled in the art.

Functionally equivalent sequences which encode the nucleic acids used in the method according to the invention are those derivatives of the sequences according to the invention which, despite deviating nucleotide sequence, retain the desired functions, that is to say the biological activity of the proteins. Functional equivalents thus encompass naturally occurring variants of the sequences described herein, and also artificial nucleotide sequences, for example artificial nucleotide sequences which have been obtained by chemical synthesis and which are, in particular, adapted to the codon usage of a plant.

Furthermore suitable are artificial DNA sequences as long as, as described above, they lead to products which mediate the abovementioned activities or the desired property, for example binding to a receptor or the enzymatic activity. Such artificial DNA sequences can be determined, for example, by backtranslating proteins which have been constructed by means of molecular modeling, or by in vitro selection. Possible techniques for the in-vitro evolution of DNA for modifying or improving the DNA sequences are described by Patten, P. A. et al., Current Opinion in Biotechnology 8, 724-733(1997) or by Moore, J. C. et al., Journal of Molecular Biology 272, 336-347( 1997). Especially suitable are coding DNA sequences which are obtained by backtranslating a polypeptide sequence in accordance with the codon usage which is specific for the host plant. The specific codon usage can be determined readily by a skilled worker who is familiar with plant genetic methods by means of computer evaluations of other, known genes of the plant to be transformed.

Amino acid sequences which are to be understood as advantageous for the method according to the invention are those comprising an amino acid sequence shown in sequences SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 75, SEQ ID NO: 77, SEQ ID NO: 79, SEQ ID NO: 81, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 99, SEQ ID NO: 101, SEQ ID NO: 103, SEQ ID NO: 105, SEQ ID NO: 107 or SEQ ID NO: 109 or a sequence which can be obtained from these by substitution, inversion, insertion or deletion of one or more amino acid residues, the biological activity of the protein shown in SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 75, SEQ ID NO: 77, SEQ ID NO: 79, SEQ ID NO: 81, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 99, SEQ ID NO: 101, SEQ ID NO: 103, SEQ ID NO: 105, SEQ ID NO: 107 oder SEQ ID NO: 109 being retained or not being reduced substantially. The term not substantially reduced refers to all those proteins which retain at least 10%, preferably 20%, especially preferably 30%, 50%, 70%, 90% or more of the biological activity of the original protein. In this context, particular amino acids can, for example, be replaced by those with similar physicochemical properties (spatial arrangement, basicity, hydrophobicity and the like). For example, arginine residues are exchanged for lysin residues, valin residues for isoleucin residues or aspartate residues for glutamate residues. However, a sequence of one or more amino acids may also be swapped, one or more amino acids may be added or removed, or several of these measures can be combined with each other.

Derivatives are also to be understood as meaning functional equivalents which encompass in particular also natural or artificial mutations of the nucleic acid sequences SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, SEQ ID NO: 78, SEQ ID NO: 80, SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 98, SEQ ID NO: 100, SEQ ID NO: 102, SEQ ID NO: 106 or SEQ ID NO: 108 used herein, which retain the desired function, that is to say that their biological activity is not substantially reduced. Mutations encompass substitutions, additions, deletions, exchanges or insertions of one or more nucleotide residues. Thus, the present invention encompasses, for example, also those nucleotide sequences which are obtained by modifying the abovementioned nucleotide sequences. The aim of such a modification can be, for example, the further delimitation of the coding sequence comprised therein or else, for example, the insertion of further cleavage sites for restriction enzymes.

Functional equivalents are also those variants whose function, compared with the original gene or gene fragment, is weakened (=not substantially reduced) or increased (=enzyme activity greater than the activity of the original enzyme, that is to say the activity is higher than 100%, preferably higher than 150%, especially preferably higher than 180%).

In this context, the nucleic acid sequence can advantageously be, for example, a DNA or cDNA sequence. Coding sequences which are suitable for insertion into a nucleic acid construct according to the invention (=expression cassette or nucleic acid fragment) are, for example, those which encode a protein with the above-described sequences and which impart, to the host, the ability to overproduce the protein and thus its biological function. These sequences can be of homologous or heterologous origin.

The invention therefore furthermore relates to a nucleic acid construct containing a nucleic acid sequence selected, for example, from the group consisting of:
a) a nucleic acid sequence with the sequence shown in SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 26, SEQ ID NO: 28, SEQ ID NO: 30, SEQ ID NO: 32, SEQ ID NO: 34, SEQ ID NO: 36, SEQ ID NO: 38 or SEQ ID NO: 40, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, SEQ ID NO: 78, SEQ ID NO: 80, SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 98, SEQ ID NO: 100, SEQ ID NO: 102, SEQ ID NO: 106 or SEQ ID NO: 108; or
b) a nucleic acid sequence which can be derived from the amino acid sequences shown in SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 75, SEQ ID NO: 77, SEQ ID NO: 79, SEQ ID NO: 81, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 99, SEQ ID NO: 101, SEQ ID NO: 103, SEQ ID NO: 105, SEQ ID NO: 107 oder SEQ ID NO: 109 by backtranslation owing to the degeneracy of the genetic code;
c) a nucleic acid sequence which is a derivative or a fragment of the nucleic acid sequences shown in SEQ ID NO: 1, SEQ ID NO: 3, SEQ ID NO: 5, SEQ ID NO: 7 SEQ ID NO: 9, SEQ ID NO: 11, SEQ ID NO: 13, SEQ ID NO: 15, SEQ ID NO: 17, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, SEQ ID NO: 78, SEQ ID NO: 80, SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 98, SEQ ID NO: 100, SEQ ID NO: 102, SEQ ID NO: 106 or SEQ ID NO: 108 and which has at least 60% homology at the nucleic acid level;
d) a nucleic acid sequence which encodes derivatives or fragments of the polypeptides with the amino acid sequences shown in SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 75, SEQ ID NO: 77, SEQ ID NO: 79, SEQ ID NO: 81, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 99, SEQ ID NO: 101, SEQ ID NO: 103, SEQ ID NO: 105, SEQ ID NO: 107 or SEQ ID NO: 109 and which have at least 50% homology at the amino acid level;
e) a nucleic acid sequence which encodes a fragment or an epitope of a polypeptide which binds specifically to an antibody, the antibody specifically binding to a polypeptide which is encoded by the sequence shown in SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, SEQ ID NO: 78, SEQ ID NO: 80, SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 98, SEQ ID NO: 100, SEQ ID NO: 102, SEQ ID NO: 106 oder SEQ ID NO: 108;
f) a nucleic acid sequence which encodes a fragment of a nucleic acid shown in a) and which has an m6A-methyltransferase activity, a DNA-binding activity or DNA repair activity, for example as in the case of RAD 54, a thioredoxin activity, a VAV2 activity, a fructokinase activity, a zinc finger protein activity, an LYTB activity, a crepopin activity, a leucine protein activity, a DNAJ activity, a CRS1 activity, an alanyl-tRNA-synthetase activity, an OEP86 activity, an FMRF amide propeptide isolog activity, a 26S proteosome subunit S5B activity or a geranylgeranyl-pyrophosphate synthase activity, cecropin family signature, ftsH chloroplast protease activity, AIM1 activity, UDP-glucuronyl transferase activity, FPF1 activity, SHI-like zinc finger protein activity, Crp1 activity, CRS1 activity, translation releasing factor RF-1 activity, farnesyl transferase subunit A activity, ATP-dependent copper transporter RAN1 activity, syntaxin or syntaxin-like-protein activity, inositol-polyphosphate-5′-phosphatase activity, UDP—N-acetylmuramoylalanyl-D-glutamate-2,6-diaminopimelate ligase activity (murE), β-glucosidase activity, hydroxymethylglutaryl-CoA reductase activity, GDSL-motif-lipase/hydroxylase-like-protein activity, cellulose-synthase-like-protein activity, tRNA-glutamine synthetase activity, exonuclease-like-protein activity, sec-independent translocase protein TATC activity or selenium-binding-protein-like-protein activity; and/or
g) a nucleic acid sequence which encodes derivatives of the polypeptides with the amino acid sequences shown in SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 75, SEQ ID NO: 77, SEQ ID NO: 79, SEQ ID NO: 81, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 99, SEQ ID NO: 101, SEQ ID NO: 103, SEQ ID NO: 105, SEQ ID NO: 107 or SEQ ID NO: 109 and which has at least 20% homology at the amino acid level and has an equivalent biological activity;
the nucleic acid sequence being linked to one or more regulatory signals. The abovementioned terms have the abovementioned meanings.

The nucleic acid construct according to the invention is to be understood as meaning the sequences stated in SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, SEQ ID NO: 78, SEQ ID NO: 80, SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 98, SEQ ID NO: 100, SEQ ID NO: 102, SEQ ID NO: 106 or SEQ ID NO: 108 as the result of the genetic code and/or their functional or nonfunctional derivatives which were functionally linked to one or more regulatory signals advantageously for regulating, in particular for increasing gene expression and which govern the expression of the coding sequence in the host cell. These regulatory sequences are intended to make possible the targeted expression of the genes or proteins. Depending on the host organism, this may mean, for example, that the gene is expressed and/or overexpressed only after induction, or that it is expressed and/or overexpressed immediately. For example, these regulatory sequences take the form of sequences to which inductors or repressors bind, thus regulating the expression of the nucleic acid. In addition to these novel regulatory sequences, or instead of these sequences, the natural regulation of these sequences may still be present before the actual structural genes and, if appropriate, have been modified genetically so that the natural regulation has been switched off and the expression of the genes increased. The nucleic acid construct according to the invention may also advantageously only be composed of the natural recombinantly modified regulatory region at the 5′ and/or 3′ end. However, the gene construct may also be constructed in a simpler fashion, that is to say no additional regulatory signals were inserted before the nucleic acid sequence or its derivatives and the natural promoter with its regulation was not removed. Instead, the natural regulatory sequence was mutated so that regulation no longer takes place and/or gene expression is increased. To increase the activity, these modified promoters may also be introduced before the natural gene by themselves in the form of part-sequences (=promoter with portions of the nucleic acid sequences according to the invention). Moreover, the gene construct can advantageously also comprise one or more of what are known as “enhancer sequences” operably linked to the promoter, and these make possible an increased expression of the nucleic acid sequence. Additional advantageous sequences such as further regulatory elements or terminators may also be inserted at the 3′ end of the DNA sequences. The nucleic acid sequences used in the method according to the invention may be present in the expression cassette (=gene construct) in one or more copies.

As described above, the regulatory sequences or factors can preferably exert a positive effect on, and thus increase, the gene expression of the genes which have been introduced. Thus, an enhancement of the regulatory elements may advantageously take place at the transcription level, by using strong transcription signals such as promoters and/or enhancers. In addition, however, increased translation is also possible, for example by improving the stability of the mRNA. In another advantageous embodiment, however, expression may also be reduced or blocked in a targeted fashion.

Promoters which are suitable as promoters in the expression cassette are, in principle, all those which are capable of governing the expression of foreign genes in organisms, advantageously in plants or fungi. In particular plant promoters or promoters originating from a plant virus are used by preference. Advantageous regulatory sequences for the method according to the invention are present, for example, in promoters such as the cos, tac, trp, tet, trp-tet, lpp, lac, lpp-lac, lacI^{q}, T7, T5, T3, gal, trc, ara, SP6, λ-P_{R} or in the λ-P_{L} promoter, these promoters being used advantageously in Gram-negative bacteria. Further advantageous regulatory sequences are present, for example, in the Gram-positive promoters amy and SPO2, in the yeast or fungal promoters ADC1, MFα, AC, P-60, CYC1, GAPDH, TEF, rp28, ADH or in the plant promoters such as in the CaMV/35S [Franck et al., Cell 21(1980) 285-294], SSU, OCS, lib4, STLS1, B33, nos (=nopaline synthase promoter) or in the ubiquitin promoter. The expression cassette may also comprise a chemically inducible promoter by which the expression of the nucleic acid sequences in the nucleic acid construct according to the invention can be controlled in the organisms, advantageously in the plants, at a particular point in time. Such advantageous plant promoters are, for example, the PRP1 promoter [Ward et al., Plant. Mol. Biol. 22(1993), 361-366), a benzenesulfonamide-inducible promoter (EP 388186), a tetracyclin-inducible promoter (Gatz et al., (1992) Plant J. 2,397-404), a salicylic-acid-inducible promoter (WO 95/19443), an abscisic-acid-inducible promoter (EP335528) or an ethanol- or cyclohexanone-inducible promoter (WO93/21334). Further plant promoters are, for example, the potato cytosolic FBPase, the potato ST-LSI promoter (Stockhaus et al., EMBO J. 8 (1989) 2445-245), the Glycine max phosphoribosyl-pyrophosphate amidotransferase promoter (see also Genbank Accession Number U87999) or a node-specific promoter such as in EP 249676 can advantageously be used.

As described above, further genes to be introduced into the organisms may also be present in the expression cassette (=gene construct, nucleic acid construct). These genes can be subject to separate regulation or subject to the same regulatory region as the nucleic acid sequences used in the method. For example, these genes take the form of biosynthesis genes of the metabolism, such as of fatty acid, amino acid or vitamin biosynthesis, or regulatory genes, to mention just a few.

In principle, all natural promoters together with their regulatory sequences, such as those mentioned above, can be used for the expression cassette according to the invention and for the method according to the invention, as described hereinbelow. Moreover, synthetic promoters may also be used advantageously.

When preparing an expression cassette, various DNA fragments can be manipulated in order to obtain a nucleotide sequence which expediently reads in the correct direction and is equipped with a correct reading frame. To connect the DNA fragments (=nucleic acids according to the invention) to each other, adapters or linkers may be attached to the fragments.

The promoter and terminator regions can expediently be provided, in the direction of transcription, with a linker or polylinker containing one or more restriction sites for the insertion of this sequence. As a rule, the linker has 1 to 10, in most cases 1 to 8, preferably 2 to 6, restriction sites. In general, the linker within the regulatory regions has a size of less than 100 bp, frequently less than 60 bp, but at least 5 bp. The promoter can be both native, or homologous, and foreign, or heterologous, with regard to the host organism, for example the host plant. In the 5′-3′ direction of transcription, the expression cassette comprises the promoter, a DNA sequence which encodes the proteins used in the method according to the invention, and a region for transcriptional termination. Various termination regions can advantageously be exchanged for each other.

Furthermore, manipulations which provide suitable restriction cleavage sites or which remove surplus DNA or restriction cleavage sites may be employed. Where insertions, deletions or substitutions such as, for example, transitions and transversions are suitable, in vitro mutagenesis, primer repair, restriction or ligation may be used. In the case of suitable manipulations such as, for example, restriction, chewing back or filling in overhangs for blunt ends, complementary ends of the fragments may be provided for ligation.

Attaching the specific ER retention signal SEKDEL (Schouten, A. et al., Plant Mol. Biol. 30 (1996), 781-792) may, inter alia, be of importance for an advantageous high level of expression; the average expression level is tripled to quadrupled thereby. Other retention signals which occur naturally in vegetable and animal proteins located in the ER may also be employed for synthesizing the cassette.

Preferred polyadenylation signals are plant polyadenylation signals, preferably those which essentially correspond to T-DNA polyadenylation signals from Agrobacterium tumefaciens, in particular of gene 3 of the T-DNA (octopine synthase) of the Ti plasmid pTiACH5 (Gielen et al., EMBO J. 3 (1984), 835 et seq.) or suitable functional equivalents.

An expression cassette is generated by fusing a suitable promoter to a suitable nucleic acid sequence and a polyadenylation signal, using customary recombination and cloning techniques as are described, for example, in T. Maniatis, E. F. Fritsch and J. Sambrook, Molecular Cloning: A Laboratory Manual, Cold Spring Harbor Laboratory, Cold Spring Harbor, N.Y. (1989) and in T. J. Silhavy, M. L. Berman and L. W. Enquist, Experiments with Gene Fusions, Cold Spring Harbor Laboratory, Cold Spring Harbor, N.Y. (1984) and in Ausubel, F. M. et al., Current Protocols in Molecular Biology, Greene Publishing Assoc. and Wiley-Interscience (1987).

When preparing an expression cassette, various DNA fragments may be manipulated in order to obtain a nucleotide sequence which expediently reads in the correct direction and which is equipped with a correct reading frame. To link the DNA fragments to each other, adapters or linkers may be attached to the fragments.

The nucleic acid sequences used in the method according to the invention encompass all sequence characteristics which are necessary to achieve a localization which is correct for the site of the biological action or activity. Thus, further targeting sequences are not necessary per se. However, such a localization may be desirable and advantageous and may therefore be modified or enhanced artificially so that such fusion constructs are also a preferred advantageous embodiment of the invention.

Advantageous for this purpose are, for example, sequences which ensure targeting into the plastids. Under certain circumstances, targeting into other compartments (reviewed in: Kermode, Crit. Rev. Plant Sci. 15, 4 (1996), 285-423), for example into the vacuole, into the mitochondrion, into the endoplasmic reticulum (ER), peroxisomes, lipid bodies or else, owing to the absence of suitable operative sequences, remaining in the compartment of formation, the cytosol, may also be desirable.

Advantageously, the nucleic acid sequences according to the invention, together with at least one reporter gene, are cloned into an expression cassette which is introduced into the organism via a vector or directly into the genome. This reporter gene should allow easy detectability via a growth, fluorescence, chemoluminescence, bioluminescence or resistance assay or via a photometric measurement. Examples of reporter genes which may be mentioned are genes for resistance to antibiotics or herbicides, hydrolase genes, fluorescent protein genes, bioluminescence genes, sugar or nucleotide metabolism genes, or biosynthesis genes such as the Ura3 gene, the Ilv2 gene, the luciferase gene, the β-galactosidase gene, the gfp gene, the β-deoxyglucose-6-phosphate phosphatase gene, the β-glucuronidase gene, the β-lactamase gene, the neomycin phosphotransferase gene, the hygromycin phosphotransferase gene, or the gene for BASTA (=glufosinate) resistance. These genes allow the transcription activity, and thus gene expression, to be measured and quantified readily. This makes possible the identification of sites in the genome which show different productivity.

In a preferred embodiment, an expression cassette comprises upstream, i.e. at the 5′ end of the coding sequence, a promoter and downstream, i.e. at the 3′ end, a polyadenylation signal and, if appropriate, further regulatory elements which are linked operably to the interposed coding sequence for the proteins used in the method according to the invention. Operable linkage is to be understood as meaning the sequential arrangement of the promoter, coding sequence, terminator and, if appropriate, further regulatory elements in such a way that each of the regulatory elements can fulfil its intended function upon expression of the coding sequence. The sequences which are preferred for operable linkage are targeting sequences for ensuring subcellular localization in the plastids. However, targeting sequences for ensuring subcellular localization in the mitochondrion, in the endoplasmic reticulum (=ER), in the nucleus, in elaioplasts or other compartments may be used, if required, or else translation enhancers such as the tobacco mosaic virus 5′ leader sequence (Gallie et al., Nucl. Acids Res. 15 (1987), 8693-8711).

An expression cassette may, for example, comprise a constitutive promoter, for example the 35S promoter, the gene to be expressed, and the ER retention signal. The amino acid sequence KDEL (lysine, aspartic acid, glutamic acid, leucin) is preferably used as ER retention signal.

For expression in a prokaryotic or eukaryotic host organism, for example a microorganism such as a fungus, or a plant, the expression cassette is advantageously inserted into a vector such as, for example, a plasmid, a phage or other DNA which makes possible optimal expression of the genes in the host organism. Suitable plasmids are, for example, in E. coli pLG338, pACYC184, pBR series, such as, for example, pBR322, pUC series, such as pUC18 or pUC19, M113mp series, pKC30, pRep4, pHS1, pHS2, pPLc236, pMBL24, pLG200, pUR290, pIN—III¹¹³—B1, λgt11 or pBdCI, in Streptomyces pIJ101, pIJ364, pIJ702 or pIJ361, in Bacillus pUB110, pC194 or pBD214, in Corynebacterium pSA77 or pAJ667, in fungi pALS1, pIL2 or pBB116, further advantageous fungal vectors are described by Romanos, M. A. et al., [(1992) “Foreign gene expression in yeast: a review”, Yeast 8: 423-488] and by van den Hondel, C. A. M. J. J. et al. [(1991) “Heterologous gene expression in filamentous fungi] and in More Gene Manipulations in Fungi [J. W. Bennet & L. L. Lasure, eds., p. 396-428: Academic Press: San Diego] and in “Gene transfer systems and vector development for filamentous fungi” [van den Hondel, C. A. M. J. J. & Punt, P. J. (1991) in: Applied Molecular Genetics of Fungi, Peberdy, J. F. et al., eds., p. 1-28, Cambridge University Press: Cambridge]. Advantageous yeast promoters are, for example, 2 μM, pAG-1, YEp6, YEp13 or pEMBLYe23. Examples of algal or plant promoters are pLGV23, pGHlac⁺, pBIN19, pAK2004, PVKH or pDH51 (see Schmidt, R. and Willmitzer, L., 1988). The abovementioned vectors or derivatives of the abovementioned vectors constitute a small selection of the plasmids which are possible. Further plasmids are well known to the skilled worker and can be found, for example, in the book Cloning Vectors (Eds. Pouwels P. H. et al. Elsevier, Amsterdam-New York-Oxford, 1985 , ISBN 0 444 904018). Suitable plant vectors are described, inter alia, in “Methods in Plant Molecular Biology and Biotechnology” (CRC Press), chapter 6/7, pp. 71-119. Advantageous vectors are what are-known as shuttle vectors or binary vectors, which replicate in E. coli and Agrobacterium.

In addition to plasmids, vectors are also to be understood as meaning all of the other vectors known to the skilled worker, such as, for example, phages, viruses such as SV40, CMV, baculovirus, adenovirus, transposons, IS elements, phasmids, phagemids, cosmids, linear or circular DNA. These vectors can be replicated autonomously in the host organism or can be replicated chromosomally; chromosomal replication is preferred. Functional and nonfunctional vectors are encompassed.

In a further embodiment of the vector, the nucleic acid construct according to the invention may also advantageously be introduced into the organisms in the form of a linear DNA and integrated into the genome of the host organism via heterologous or homologous recombination. This linear DNA may be composed of a linearized plasmid or only of the nucleic acid construct as vector, or the nucleic acid sequences used.

In a further advantageous embodiment, the nucleic acid sequences used in the method according to the invention may also be introduced into an organism by themselves.

If, in addition to the nucleic acid sequences, further genes are to be introduced into the organism, all may be introduced into the organism together with a reporter gene in a single vector, or each individual gene with or without a reporter gene in a separate vector, it being possible to introduce the various vectors simultaneously or in succession.

The vector advantageously comprises at least one copy of the nucleic acid sequences used and/or of the nucleic acid construct according to the invention.

For example, the nucleic acid construct can be incorporated into the tobacco transformation vector pBinAR and be under the control of the 35S promoter or the USP promoter.

As an alternative, a recombinant vector (=expression vector) may also be transcribed and translated in vitro, for example by using the T7 promoter and T7 RNA polymerase.

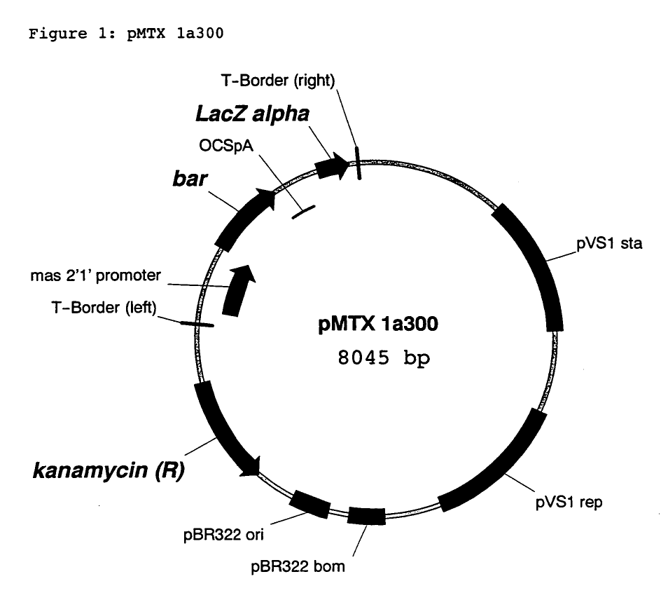

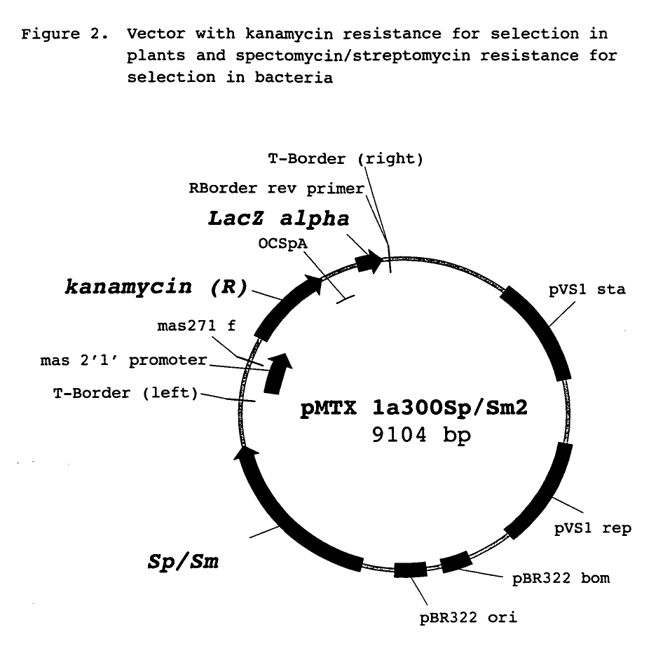

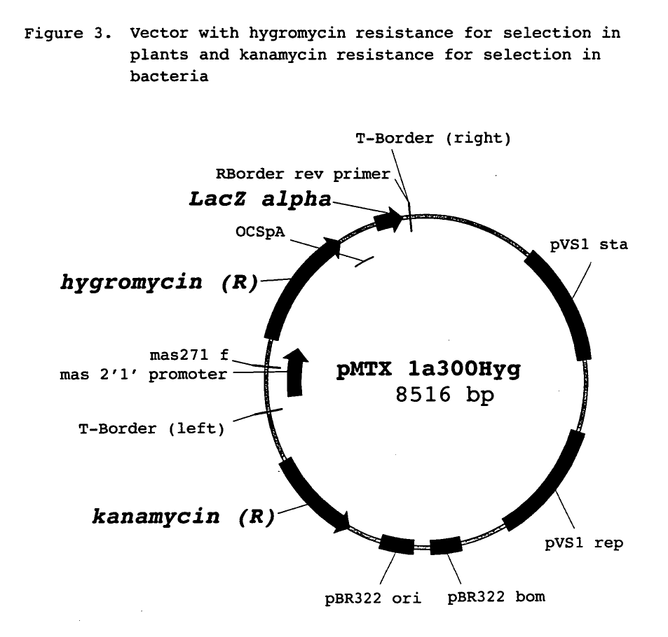

Further advantageous vectors comprise resistances which can be used in plants or plant crops, such as the resistance to phosphinothricin (=bar resistance), the resistance to methionine sulfoximine, the resistance to sulfonylurea (=i1v resistance, S. cerevisiae i1v2), the resistance to phenoxyphenoxy herbicide (=ACCase resistance), the resistance to glyphosate or Clearfield (AHAS resistance), or the genes which encode these resistances. These resistances can be exploited in intact plants for selecting transgenic plants. Only plants to which these resistances have been imparted via a transformation process are capable of growing in the presence of the selecting substance. Following transformation in planta—for example infiltration of the seed precursor cells—kanamycin or hygromycin are other examples of selecting agents in cell cultures on agar plates. Moreover, advantageous vectors may comprise sequences for integration into the genome of the organisms, preferably the plants. Examples of such sequences are what are known as T-DNA borders. In addition, advantageous vectors may also comprise promoters and terminators such as, for example, those described above. What are known as poly-A sequences may also be present in the vector. Advantageous vectors can be found, for example, in FIGS. 1, 2 and 3. SEQ ID NO: 25 indicates the advantageous sequence of vector pMTX 1a300. This vector contains a kanamycin resistance (nucleotide 4922-5713), a phosphinothricin resistance (nucleotide 6722-7288), the LacZalpha fragment (nucleotide 7630-7864), a portion of pVS1sta (nucleotide 945-1945), a portion of pBR322bom (nucleotide 3948-4208), a T border sequence (left, nucleotide 6138-6163), a T border sequence (right, nucleotide 7924-7949), a poly-A portion (nucleotide 7292-7503), the mas2′1′ promoter (nucleotide 6241-6718) and two origins of replication pVS1rep (nucleotide 6241-6718) and pBR322ori (nucleotide 43-4628).

Expression vectors used in prokaryotes frequently exploit inducible systems with and without fusion proteins or fusion oligopeptides, it being possible for these fusions to be effected at the N terminal or the C terminal or other utilizable domains of a protein. In general, the purpose of such fusion vectors is: i.) to increase the expression rate of the RNA, ii.) to increase the achievable protein synthesis rate, iii.) to increase the solubility of the protein, or iv.) to simplify purification by a binding sequence which can be exploited in affinity chromatography. Also, proteolytic cleavage sites are frequently introduced via fusion proteins, which makes possible the elimination of a portion of the fusion protein upon purification. Such recognition sequences which proteases recognize are, for example, factor Xa, thrombin and enterokinase.

Typical advantageous fusion and expression vectors are pGEX [Pharmacia Biotech Inc; Smith, D. B. and Johnson, K. S. (1988) Gene 67:31-40], pMAL (New England Biolabs, Beverly, Mass.) and pRIT5 (Pharmacia, Piscataway, N.J.), which comprises glutathione S transferase (GST), maltose binding protein, or protein A.

Further examples for E. coli expression vectors are pTrc [Amann et al., (1988) Gene 69:301-315] and pET vectors [Studier et al., Gene Expression Technology: Methods in Enzymology 185, Academic Press, San Diego, Calif. (1990) 60-89; Stratagene, Amsterdam, Netherlands].

Further advantageous vectors for use in yeast are pYepSec1 (Baldari, et al., (1987) Embo J. 6:229-234), pMFa (Kurjan and Herskowitz, (1982) Cell 30:933-943), pJRY88 (Schultz et al., (1987) Gene 54:113-123), and pYES derivatives (Invitrogen Corporation, San Diego, Calif.). Vectors for use in filamentous fungi are described in: van den Hondel, C. A. M. J. J. & Punt, P. J. (1991) “Gene transfer systems and vector development for filamentous fungi, in: Applied Molecular Genetics of Fungi, J. F. Peberdy, et al., eds., p. 1-28, Cambridge University Press: Cambridge.

As an alternative, insect cell expression vectors may also be used advantageously, for example for expression in Sf 9 cells. Examples of these are the vectors of the pAc series (Smith et al. (1983) Mol. Cell Biol. 3:2156-2165) and of the pVL series (Lucklow and Summers (1989) Virology 170:31-39).

Moreover, plant cells or algal cells may advantageously be used for gene expression. Examples of plant expression vectors are found in Becker, D., et al. (1992) “New plant binary vectors with selectable markers located proximal to the left border”, Plant Mol. Biol. 20: 1195-1197 or in Bevan, M. W. (1984) “Binary Agrobacterium vectors for plant transformation”, Nucl. Acid. Res. 12: 8711-8721.

Furthermore, the nucleic acid sequences according to the invention can be expressed in mammalian cells. Examples of suitable expression vectors are pCDM8 and pMT2PC, which are mentioned in: Seed, B. (1987) Nature 329:840 or Kaufman et al. (1987) EMBO J. 6:187-195). Promoters preferably to be used are of viral origin, such as, for example, promoters of polyoma virus, adenovirus 2, cytomegalovirus or simian virus 40. Further prokaryotic and eukaryotic expression systems are mentioned in chapters 16 and 17 in Sambrook et al., Molecular Cloning: A Laboratory Manual. 2nd, ed., Cold Spring Harbor Laboratory, Cold Spring Harbor Laboratory Press, Cold Spring Harbor, N.Y., 1989. Further advantageous vectors are described in Hellens et al. (Trends in plant science, 5, 2000).

In principle, the nucleic acids according to the invention, the expression cassette or the vector can be introduced into organisms, for example into plants, by all methods with which the skilled worker is familiar.

For microorganisms, the skilled worker will find suitable methods in the textbooks by Sambrook, J. et al. (1989) Molecular cloning: A laboratory manual, Cold Spring Harbor Laboratory Press, by F. M. Ausubel et al. (1994) Current protocols in molecular biology, John Wiley and Sons, by D. M. Glover et al., DNA Cloning Vol.1, (1995), IRL Press (ISBN 019-963476-9), by Kaiser et al. (1994) Methods in Yeast Genetics, Cold Spring Habor Laboratory Press or Guthrie et al. Guide to Yeast Genetics and Molecular Biology, Methods in Enzymology, 1994, Academic Press.

The transfer of foreign genes into the genome of a plant is referred to as transformation. It exploits the above-described methods of transforming and regenerating plants from plant tissues or plant cells for transient or stable transformation. Suitable methods are protoplast transformation by polyethylene glycol-induced DNA uptake, the biolistic method with the gene gun—known as the particle bombardment method—, electroporation, incubation of dry embryos in DNA-containing solution, microinjection and Agrobacterium-mediated gene transfer. In the present invention, the gene transfer is advantageously effected using, for example, Agrobacterium tumefaciens strain GV 3101 pMP90. The abovementioned methods are described in, for example, B. Jenes et al., Techniques for Gene Transfer, in: Transgenic Plants, Vol. 1, Engineering and Utilization, edited by S. D. Kung and R. Wu, Academic Press (1993) 128-143 and in Potrykus Annu. Rev. Plant Physiol. Plant Molec. Biol. 42 (1991) 205-225. The construct to be expressed is preferably cloned into a vector which is suitable for transforming Agrobacterium tumefaciens, for example pBin19 (Bevan et al., Nucl. Acids Res. 12 (1984) 8711). Agrobacteria transformed with such a vector can then be used for transforming plants, in particular crop plants such as, for example, tobacco plants, in the known manner, for example by bathing scarified leaves or leaf sections in an agrobacterial solution and subsequently growing them in suitable media. The transformation of plants with Agrobacterium tumefaciens is described, for example, by Höfgen and Willmitzer in Nucl. Acid Res. (1988) 16, 9877 or is known, inter alia, from F. F. White, Vectors for Gene Transfer in Higher Plants; in Transgenic Plants, Vol. 1, Engineering and Utilization, edited by S. D. Kung and R. Wu, Academic Press, 1993, pp. 15-38.

An advantageous embodiment is described hereinbelow. If agrobacteria are used for the transformation, the nucleic acid or DNA to be introduced will be cloned into specific plasmids, either into an intermediary vector or into a binary vector. The intermediary vectors can be integrated into the Ti or Ri plasmid of the agrobacteria by homologous recombination, owing to sequences which are homologous to sequences in the T-DNA. The Ti or Ri plasmid additionally comprises the vir region, which is required for the transfer of the T-DNA. Intermediary vectors are not capable of replication in agrobacteria. The intermediary vector can be transferred to Agrobacterium tumefaciens by means of a helper plasmid (conjugation). Binary vectors are capable of replication both in E. coli and in agrobacteria. They comprise a selection marker gene and a linker or polylinker, which are framed by the right and left T-DNA border region. They can be transformed directly into the agrobacteria (Holsters et al. Mol. Gen. Genet. 163 (1978), 181-187). The agrobacterium which acts as the host cell should comprise a plasmid carrying a vir region. The vir region is required for the transfer of the T-DNA into the plant cell. Additional T-DNA may be present. The agrobacterium transformed in this way is used for transforming plant cells.

The use of T-DNA for transforming plant cells has been studied intensively and described amply in EPA-0 120 516; Hoekema, In: The Binary Plant Vector System Offsetdrukkerij Kanters B. V., Alblasserdam (1985), Chapter V; Fraley et al., Crit. Rev. Plant. Sci., 4: 1-46 and An et al. EMBO J. 4 (1985), 277-287.

To transfer the DNA into the plant cell, plant explants can expediently be cocultured with Agrobacterium tumefaciens or Agrobacterium rhizogenes. Then, intact plants can be regenerated from the infected plant material (for example leaf sections, stem segments, roots, but also protoplasts, or plant cells grown in suspension culture) in a suitable medium which may comprise antibiotics or biocides for selecting transformed cells. The plants obtained in this way can then be examined for the presence of the DNA introduced. Other possibilities of introducing foreign DNA using the biolistic method or by protoplast transformation are known (cf., for example, Willmitzer, L., 1993 Transgenic plants. In: Biotechnology, A Multi-Volume Comprehensive Treatise (H. J. Rehm, G. Reed, A. Pühler, P. Stadler, eds.), Vol. 2, 627-659, VCH Weinheim-New York-Basel-Cambridge).

The transformation of monocotyledonous plants by means of Agrobacterium-based vectors has also been described (Chan et al, Plant Mol. Biol. 22(1993), 491-506; Hiei et al, Plant J. 6 (1994) 271-282; Deng et al.; Science in China 33 (1990), 28-34; Wilmink et al., Plant Cell Reports 11,(1992) 76-80; May et al.; Biotechnology 13 (1995) 486-492; Conner and Domisse; Int. J. Plant Sci. 153 (1992) 550-555; Ritchie et al.; Transgenic Res. (1993) 252-265). Alternative systems for transforming monocotyledonous plants are the transformation by means of the biolistic approach (Wan and Lemaux; Plant Physiol. 104 (1994), 37-48; Vasil et al.; Biotechnology 11 (1992), 667-674; Ritala et al., Plant Mol. Biol 24, (1994) 317-325; Spencer et al., Theor. Appl. Genet. 79 (1990), 625-631), protoplast transformation, the electroporation of partially permeabilized cells, the introduction of DNA by means of glass fibers. In particular the transformation of maize has been described repeatedly in the literature (cf., for example, WO 95/06128; EP 0513849 A1; EP 0465875 A1; EP 0292435 A1; Fromm et al., Biotechnology 8 (1990), 833-844; Gordon-Kamm et al., Plant Cell 2 (1990), 603-618; Koziel et al., Biotechnology 11(1993) 194-200; Moroc et al., Theor Applied Genetics 80 (190) 721-726).

The successful transformation of other cereal species has also been described, for example in the case of barley (Wan and Lemaux, see above;) Ritala et al., see above; wheat (Nehra et al., Plant J. 5(1994) 285-297).

Agrobacteria transformed with a vector according to the invention can also be used in the known manner for transforming plants such as test plants such as Arabidopsis or crop plants such as cereals, maize, oats, rye, barley, wheat, soybean, rice, cotton, sugar beet, canola, sunflower, flax, hemp, potato, tobacco, tomato, carrot, capsicum, oilseed rape, tapioca, cassava, arrowroot, marigolds, alfalfa, lettuce and the various tree, nut and grapevine species, for example by bathing scarified leaves or leaf segments in an agrobacterial solution and subsequently growing them in suitable media.

The genetically modified plant cells can be regenerated via all methods known to the skilled worker. Suitable methods can be found in the abovementioned publications by S. D. Kung and R. Wu, Potrykus or Höfgen and Willmitzer.

For the purposes of the invention, plants are to be understood as meaning plant cells, plant tissue, plant organs or intact plants such as seeds, tubers, flowers, pollen, fruits, seedlings, roots, leaves, stems or other plant parts. Moreover, plants are to be understood as meaning propagation material such as seeds, fruits, seedlings, slips, tubers, cuttings or rootstocks.

In principle, suitable organisms or host organisms for the nucleic acid according to the invention, the expression cassette or the vector are advantageously all organisms which are capable of expressing the nucleic acids used in accordance with the invention or which are suitable for the expression of recombinant genes. Plants which may be mentioned by way of example are Arabidopsis, Asteraceae such as Calendula, or crop plants such as soybean, peanut, castor-oil plant, sunflower, maize, cotton, flax, oilseed rape, coconut, oil palm, safflower (Carthamus tinctorius) or cacao, microorganisms such as fungi, for example the genus Mortierella, Saprolegnia or Pythium, bacteria such as the genus Escherichia, yeasts such as the genus Saccharomyces, cyanobacteria, ciliates, algae or protozoans such as dinoflagellates, such as Crypthecodinium. Organisms which naturally synthesize substantial amounts of oils and which may be mentioned by way of example are soybean, oilseed rape, coconut, oil palm, safflower, castor-oil plant, Calendula, peanut, cacao or sunflower. In principle, transgenic animals are also suitable as host organisms, for example C. elegans.

Preferred transgenic plants are those which comprise a functional or nonfunctional nucleic acid construct according to the invention or a functional or nonfunctional vector according to the invention. For the purposes of the invention, functional means that the nucleic acids used in the method, alone or in the nucleic acid construct or in the vector, are expressed and a biologically active gene product is produced. For the purposes of the invention, nonfunctional means that the nucleic acids used in the method, alone or in the nucleic acid construct or in the vector are not transcribed or not expressed and/or that a biologically inactive gene product is produced. In this sense, what are known as antisense RNAs are also nonfunctional nucleic acids or, upon insertion into the nucleic acid construct or the vector, a nonfunctional nucleic acid construct or nonfunctional vector. To generate transgenic organisms, preferably plants, both the nucleic acid construct according to the invention and the vector according to the invention can be used advantageously.

For the purposes of the invention, transgenic/recombinantly is to be understood as meaning that the nucleic acids used in the method are not at their natural place in the genome of an organism, it being possible for the nucleic acids to be expressed homologously or heterologously. However, transgenic/recombinantly also means that the nucleic acids according to the invention are at their natural position in the genome of an organism, but that the sequence has been modified compared with the natural sequence and/or that the regulatory sequences of the natural sequences have been modified. Preferably, transgenic/recombinantly is to be understood as meaning the expression of the nucleic acids at a non-natural position in the genome, that is to say homologous or, preferably, heterologous expression of the nucleic acids takes place. The same also applies to the nucleic acid construct according to the invention or the vector.

Utilizable host cells are furthermore mentioned in: Goeddel, Gene Expression Technology: Methods in Enzymology 185, Academic Press, San Diego, Calif. (1990).

Expression strains which can be used, for example those which exhibit a lower protease activity, are described in: Gottesman, S., Gene Expression Technology: Methods in Enzymology 185, Academic Press, San Diego, Calif. (1990) 119-128.

Furthermore, the invention also encompasses the use of the nucleic acids according to the invention, for example of the nucleotide sequences stated in SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, SEQ ID NO: 78, SEQ ID NO: 80, SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 98, SEQ ID NO: 100, SEQ ID NO: 102, SEQ ID NO: 106 or SEQ ID NO: 108 for generating genetically modified plants which comprise modified proteins of the proteins encoded by SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, SEQ ID NO: 78, SEQ ID NO: 80, SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 98, SEQ ID NO: 100, SEQ ID NO: 102, SEQ ID NO: 106 or SEQ ID NO: 108 which have a very much lower interaction with the herbicide or whose activity is not interfered with by the herbicide.

The nucleic acids used in the method according to the invention, in particular SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, SEQ ID NO: 78, SEQ ID NO: 80, SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 98, SEQ ID NO: 100, SEQ ID NO: 102, SEQ ID NO: 106 or SEQ ID NO: 108, the sequences which have been derived from them on the basis of the degeneracy of the genetic code and their derivatives were identified from a population of transgenic plants, which population has, on the one hand, been transformed by means of Agrobacterium and, while performing this process, novel DNA had been integrated randomly in the chromosome. Backcrosses finally allowed plants to be isolated which contain the identified nucleic acids on both homologous chromosomes. Moreover, these plants have been identified during the screening process as lines which segregate for lethal mutations. As a result of the integration of the novel DNA, these plants show severely impaired growth and development. It can be assumed that this impaired growth and development can be attributed to the fact that the newly inserted DNA has integrated into genes which are important for growth and development, thus limiting or blocking their biological function. This means that these genes and the sequences which have been derived on the basis of the degeneracy of the genetic code and their derivatives encode proteins which, analogously to those described in SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, SEQ ID NO: 78, SEQ ID NO: 80, SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 98, SEQ ID NO: 100, SEQ ID NO: 102, SEQ ID NO: 106 or SEQ ID NO: 108 constitute suitable target proteins for herbicides to be newly developed.

In an advantageous embodiment, the stated nucleic acids are overexpressed and the following process steps are advantageously carried out in order to generate such modified proteins:
a) expression, in a heterologous system, for example a microorganism such as a bacterium of the genus Escherichia, such as E. coli XL1-Red, or in a cell-free system, of the proteins encoded by the nucleic acid sequences shown in SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, SEQ ID NO: 78, SEQ ID NO: 80, SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 98, SEQ ID NO: 100, SEQ ID NO: 102, SEQ ID NO: 106 or SEQ ID NO: 108 or by a nucleic acid sequence which can be derived on the basis of the degeneracy of the genetic code by backtranslating the amino acid sequences shown in SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 75, SEQ ID NO: 77, SEQ ID NO: 79, SEQ ID NO: 81, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 99, SEQ ID NO: 101, SEQ ID NO: 103, SEQ ID NO: 105, SEQ ID NO: 107 or SEQ ID NO: 109 or of proteins encoded by derivatives or fragments of the nucleic acid sequences shown in SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, SEQ ID NO: 78, SEQ ID NO: 80, SEQ ID NO: 82 oder SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 98, SEQ ID NO: 100, SEQ ID NO: 102, SEQ ID NO: 106, SEQ ID NO: 108 which encode polypeptides with the amino acid sequences shown in SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 75, SEQ ID NO: 77, SEQ ID NO: 79, SEQ ID NO: 81, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 99, SEQ ID NO: 101, SEQ ID NO: 103, SEQ ID NO: 105, SEQ ID NO: 107 or SEQ ID NO: 109 and which have at least 50%, 60%, preferably 70%, 80%, 90% or more homology at the amino acid level,
b) randomized or directed mutagenesis of the protein by modification of the nucleic acid,
c) measuring the interaction or the biological activity of the modified protein with the herbicide, or in the presence of the herbicide,
d) identification of derivatives of the protein which exhibit a lesser degree of interaction or a biological activity which has been affected to a lesser degree,
e) assaying the biological activity of the protein following application of the herbicide.

The resulting modified protein, or the modified nucleic acid, for example of the sequences stated under SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 26, SEQ ID NO: 27, SEQ ID NO: 30, SEQ ID NO: 32, SEQ ID NO: 34, SEQ ID NO: 36, SEQ ID NO: 38, SEQ ID NO: 40, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, SEQ ID NO: 78, SEQ ID NO: 80, SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 98, SEQ ID NO: 100, SEQ ID NO: 102, SEQ ID NO: 106 or SEQ ID NO: 108 and the other sequences described above, for example derivatives and fragments, for example of other plants, are advantageously transferred into an organism, advantageously into a plant, preferably plant cells.

A further embodiment of the invention is a method for generating modified gene products encoded by the nucleic acid sequences SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, SEQ ID NO: 78, SEQ ID NO: 80, SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 98, SEQ ID NO: 100, SEQ ID NO: 102, SEQ ID NO: 106 oder SEQ ID NO: 108 according to the invention and described herein which comprises the following process steps:
a) expression of the proteins encoded by SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, SEQ ID NO: 78, SEQ ID NO: 80, SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 98, SEQ ID NO: 100, SEQ ID NO: 102, SEQ ID NO: 106 or SEQ ID NO: 108 or their derivatives or fragments in a heterologous system or in a cell-free system
b) randomized or directed mutagenesis of the protein by modification of the nucleic acid,
c) measuring the interaction or the biological activity of the modified gene product with the herbicide, or in the presence of the herbicide,
d) identification of derivatives of the protein which exhibit a lesser degree of interaction or whose activity is less affected,
e) assaying the biological activity of the protein following application of the herbicide,
f) selection of the nucleic acid sequences which, or whose gene products, show a modified biological activity with regard to the herbicide, preferably a reduced inhibition by the herbicide or a lesser degree of interaction with the herbicide.

The sequences selected by the above-described method are advantageously introduced into an organism. Therefore, the invention furthermore relates to an organism generated by this method, the organism preferably being a plant. The method is also suitable for the gene expression of the abovementioned biologically active derivatives and fragments.

Subsequently, intact plants are regenerated and the resistance to the herbicide is tested in intact plants.

Modified proteins and/or nucleic acids which, in plants, can mediate resistance to herbicides can also be generated from the sequences according to the invention described herein, in particular from the sequences SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, SEQ ID NO: 78, SEQ ID NO: 80, SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 98, SEQ ID NO: 100, SEQ ID NO: 102, SEQ ID NO: 106 or SEQ ID NO: 108 or their derivatives from other plants via what is known as site-directed mutagenesis. For example, the stability and/or enzymatic activity of enzymes or the properties such as the binding of low-molecular-weight compounds with less than 1000 daltons, advantageously less than 900 daltons, preferably less than 800, especially preferably less than 700, very especially preferably less than 600 daltons, of proteins or of antisense RNA can be improved or modified in a highly targeted fashion by means of this mutagenesis.

Moreover, modifications may be achieved by the PCR method described by Spee et al. (Nucleic Acids Research, Vol. 21, No. 3, 1993: 777-78), using dITP for the random mutagenesis, or by the further improved method of Rellos et al. (Protein Expr. Purif., 5, 1994 : 270-277).

A further possibility of generating these modified proteins and/or nucleic acids is the in vitro recombination technique described by Stemmer et al. (Proc. Natl. Acad. Sci. USA, Vol. 91, 1994: 10747-10751) for molecular evolution or the combination of the PCR and recombination method, which has been described by Moore et al. (Nature Biotechnology Vol. 14, 1996: 458-467).

A further way of mutating nucleic acids and proteins is described by Greener et al. in Methods in Molecular Biology (Vol. 57, 1996: 375-385). EP-A-0 909 821 describes a method of modifying proteins using the microorganism E. coli XL-1 Red. Upon replication, this microorganism generates mutations in the introduced nucleic acids and thus leads to a modification of the genetic information. Advantageous nucleic acids and the proteins encoded by them can be identified readily via isolation of the modified nucleic acids or the modified proteins and carrying out of resistance testing, and vice versa. After introduction into plants, they can manifest resistance therein and thus lead to resistance to the herbicides.

Further methods of mutagenesis and selection are, for example, methods such as the in vivo mutagenesis of seeds or pollen and selection of resistant alleles in the presence of the inhibitors according to the invention, followed by the genetic and molecular identification of the modified, resistant allele. Furthermore, the mutagenesis and selection of resistances in cell culture by propagating the culture in the presence of successively increasing concentrations of the inhibitors according to the invention. In doing so, the increase in the spontaneous mutation rate by chemical/physical mutagenic treatment may be exploited. As described above, modified genes may also be isolated using microorganisms which have an endogenous or recombinant activity of the proteins encoded by the nucleic acids used in the method according to the invention, which microorganisms are sensitive to the inhibitors identified in accordance with the invention. Growing the microorganisms on media with increasing concentrations of inhibitors according to the invention permits the selection and evolution of resistant variants of the targets according to the invention. The frequency of the mutations, in turn, can be increased by mutagenic treatments.

In addition, methods are available for the targeted modifications of nucleic acids (Zhu et al. Proc. Natl. Acad. Sci. USA, Vol. 96, 8768-8773 and Beethem et al., Proc. Natl. Acad. Sci. USA, Vol 96, 8774-8778). These methods make it possible to replace, in the proteins, those amino acids which are of importance for binding inhibitors by functionally equivalent amino acids which, however, inhibit the binding of the inhibitor.

The invention therefore furthermore relates to a method of generating nucleotide sequences which encode gene products with a modified biological activity, the biological activity being modified such that an increased activity is present. Increased activity is to be understood as meaning an activity which is increased over the original organism, or over the original gene product, by at least 10%, preferably by at least 30%, especially preferably by at least 50% or 70%, very especially preferably by at least 100%. Moreover, the biological activity may have been modified such that the substances and/or compositions according to the invention no longer, or no longer correctly, bind to the nucleic acid sequences and/or the gene products encoded by them. No longer, or no longer correctly, is to be understood as meaning for the purposes of the invention that the substances bind at least 30% less, preferably at least 50% less, especially referably at least 70% less, very especially preferably at least 80% less or not at all to the modified nucleic acids and/or gene products in comparison with the original gene product or the original nucleic acids.

Yet a further aspect of the invention therefore relates to a transgenic plant which has been genetically modified by the above-described method according to the invention.

Genetically modified transgenic plants which are resistant to the substances found in accordance with the methods according to the invention and/or to compositions comprising these substances may also be generated by overexpressing the nucleic acids, in particular with the sequences SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, SEQ ID NO: 78, SEQ ID NO: 80, SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 98, SEQ ID NO: 100, SEQ ID NO: 102, SEQ ID NO: 106 or SEQ ID NO: 108 used in the methods according to the invention. The invention therefore furthermore relates to a method of generating transgenic plants which are resistant to substances which have been found by a method according to the invention, wherein nucleic acids according to the invention with one of the above-described biological activities in particular with the sequences SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 26, SEQ ID NO: 27, SEQ ID NO: 30, SEQ ID NO: 32, SEQ ID NO: 34, SEQ ID NO: 36, SEQ ID NO: 38, SEQ ID NO: 40, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, SEQ ID NO: 78, SEQ ID NO: 80, SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 98, SEQ ID NO: 100, SEQ ID NO: 102, SEQ ID NO: 106 or SEQ ID NO: 108 are overexpressed in these plants. A similar method is described, for example, in Lermantova et al., Plant Physiol., 122, 2000: 75-83. Naturally, the derivatives and fragments mentioned herein which have the desired activity may also be used, for example those from other plants.

The above-described methods according to the invention for generating resistant plants make possible the development of novel herbicides which have as complete as possible an action which is independent of the plant species (what are known as nonselective herbicides), in combination with the development of useful plants which are resistant to the nonselective herbicide. Useful plants which are resistant to nonselective herbicides have already been described on several occasions. In this context, one can distinguish between several principles for achieving a resistance:
a) Generation of resistance in a plant via mutation methods or recombinant methods by markedly overproducing the protein which acts as target for the herbicide and by the fact that, owing to the large excess of the protein which acts as target for the herbicide, the function exerted by this protein in the cell is even retained after application of the herbicide.
b) Modification of the plant such that a modified version of the protein which acts as target of the herbicide is introduced and that the function of the newly introduced modified protein is not adversely affected by the herbicide.
c) Modification of the plant such that a novel protein/a novel RNA is introduced wherein the chemical structure of the protein or of the nucleic acid, such as of the RNA or the DNA, which structure is responsible for the herbicidal action of the low-molecular-weight substance, is modified so that, owing to the modified structure, a herbicidal action can no longer be developed or that the herbicide in the modified plant is inactivated or modified, for example catabolized, not taken up or not transported or transported into the vacuole, and the like, that is to say that the interaction of the herbicide with the target can no longer take place.
d) The function of the target is replaced by a novel nucleic acid introduced into the plant, for example a gene, the nucleic acid encoding a gene product whose function is inhibited to a lesser degree or not at all by the herbicidal substance. In this manner, for example, what is known as an alternative pathway is created.
e) The function of the target is taken over by another gene which is present in the plant or introduced into the plant, or by its gene product.

The present invention therefore furthermore relates to the use of plants comprising the genes affected by T-DNA insertion which have the nucleic acid sequences SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, SEQ ID NO: 78, SEQ ID NO: 80, SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 98, SEQ ID NO: 100, SEQ ID NO: 102, SEQ ID NO: 106 or SEQ ID NO: 108 used in the method according to the invention or the other sequences mentioned, for example fragments and derivatives, for example from other plants, for the development of novel herbicides. The skilled worker is familiar with alternative methods of identifying homologous nucleic acids, for example in other plants with similar sequences, such as, for example, using transposons. The present invention therefore also relates to the use of alternative insertion mutagenesis methods for inserting foreign nucleic acid into the nucleic acid sequences SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, SEQ ID NO: 78, SEQ ID NO: 80, SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 98, SEQ ID NO: 100, SEQ ID NO: 102, SEQ ID NO: 106 or SEQ ID NO: 108 according to the invention and described here, into sequences derived from these sequences on the basis of the genetic code and/or their derivatives or fragments, for example those from other plants.

The invention therefore furthermore relates to substances as described above, identified by the methods according to the invention, the substance having a molecular weight of less than 1000 daltons, advantageously less than 900 daltons, preferably less than 800 daltons, especially preferably less than 700 daltons, very especially preferably less than 600 daltons, a Ki value of under 1 mM and fewer than three hydroxyl groups on a carbon-atom-containing ring, the substance being a proteinogenic substance or an antisense RNA.

A further embodiment of the invention are substances which have been identified by the methods according to the invention described hereinabove, the substances being an antibody to the protein encoded by sequences SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, SEQ ID NO: 78, SEQ ID NO: 80, SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 98, SEQ ID NO: 100, SEQ ID NO: 102, SEQ ID NO: 106 or SEQ ID NO: 108 or derivatives or fragments of this protein.

The antibodies can also bind more than one of the abovementioned sequences as long as the binding is specific, i.e. capable of being identified or assayed by means of the abovementioned methods.

These substances are advantageously distinguished by their herbicidal action which can be identified by means of the above-described methods.

The invention furthermore relates to compositions comprising a herbicidally active amount of at least one substance identified by one of the methods according to the invention or of an antagonist identified by a method according to the invention, and at least one inert liquid and/or solid carrier and, if appropriate, at least one surface-active substance.

A further embodiment are compositions comprising a growth-regulatory amount of at least one substance identified by the methods according to the invention or of an antagonist identified by a method according to the invention, and at least one inert liquid and/or solid carrier and, if appropriate, at least one surface-active substance.

These substances or compositions according to the invention with their herbicidal action can be used as defoliants, desiccants, haulm killers and, in particular, as weed killers. Weeds are to be understood as meaning, in the broadest sense, all plants which grow in locations where they are undesired. Whether the substances or active ingredients found with the aid of the methods according to the invention act as nonselective or selective herbicides depends, inter alia, on the amount used, their selectivity and other factors. For example, the substances can be used against the following weeds:

Dicotyledonous weeds of the genera:
Sinapis, Lepidium, Galium, Stellaria, Matricaria, Anthemis, Galinsoga, Chenopodium, Urtica, Senecio, Amaranthus, Portulaca, Xanthium, Convolvulus, Ipomoea, Polygonum, Sesbania, Ambrosia, Cirsium, Carduus, Sonchus, Solanum, Rorippa, Rotala, Lindernia, Lamium, Veronica, Abutilon, Emex, Datura, Viola, Galeopsis, Papaver, Centaurea, Trifolium, Ranunculus, Taraxacum.

Monocotyledonous weeds of the genera:
Echinochloa, Setaria, Panicum, Digitaria, Phleum, Poa, Festuca, Eleusine, Brachiaria, Lolium, Bromus, Avena, Cyperus, Sorghum, Agropyron, Cynodon, Monochoria, Fimbristyslis, Sagittaria, Eleocharis, Scirpus, Paspalum, Ischaemum, Sphenoclea, Dactyloctenium, Agrostis, Alopecurus, Apera.

Depending on the application method in question, the substances identified in the method according to the invention, or compositions comprising them, may advantageously also be employed in a further number of crop plants for eliminating undesired plants. Examples of suitable crops are:
Allium cepa, Ananas comosus, Arachis hypogaea, Asparagus officinalis, Beta vulgaris spec. altissima, Beta vulgaris spec. rapa, Brassica napus var. napus, Brassica napus var. napobrassica, Brassica rapa var. silvestris, Camellia sinensis, Carthamus tinctorius, Carya illinoinensis, Citrus limon, Citrus sinensis, Coffea arabica (Coffea canephora, Coffea liberica), Cucumis sativus, Cynodon dactylon, Daucus carota, Elaeis guineensis, Fragaria vesca, Glycine max, Gossypium hirsutum, (Gossypium arboreum, Gossypium herbaceum, Gossypium vitifolium), Helianthus annuus, Hevea brasiliensis, Hordeum vulgare, Humulus lupulus, Ipomoea batatas, Juglans regia, Lens culinaris, Linum usitatissimum, Lycopersicon lycopersicum, Malus spec., Manihot esculenta, Medicago sativa, Musa spec., Nicotiana tabacum (N.rustica), Olea europaea, Oryza sativa, Phaseolus lunatus, Phaseolus vulgaris, Picea abies, Pinus spec., Pisum sativum, Prunus avium, Prunus persica, Pyrus communis, Ribes sylestre, Ricinus communis, Saccharum officinarum, Secale cereale, Solanum tuberosum, Sorghum bicolor (s. vulgare), Theobroma cacao, Trifolium pratense, Triticum aestivum, Triticum durum, Vicia faba, Vitis vinifera, Zea mays.

The substances found by the method according to the invention can also be used advantageously in crops which tolerate the action of herbicides owing to breeding, including recombinant methods.

The substances according to the invention, or the herbicidal compositions comprising them, can be applied, for example, in the form of directly sprayable aqueous solutions, powders, suspensions, also highly concentrated aqueous, oily or other suspensions or dispersions, emulsions, oil dispersions, pastes, dusts, materials for spreading or granules by means of spraying, atomizing, dusting, spreading or pouring. The use forms depend on the intended purposes; in any case, they should guarantee the finest possible distribution of the active ingredients according to the invention.

Suitable inert liquid and/or solid carriers are liquid additives such as mineral oil fractions of medium to high boiling point, such as kerosine or diesel oil, furthermore coal tar oils and oils of vegetable or animal origin, aliphatic, cyclic and aromatic hydrocarbons, for example paraffin, tetrahydronaphthalene, alkylated naphthalenes or their derivatives, alkylated benzenes or their derivatives, alcohols such as methanol, ethanol, propanol, butanol, cyclohexanol, ketones such as cyclohexanone or strongly polar solvents, for example amines such as N-methylpyrrolidone or water.

Further advantageous embodiments of the substances and/or compositions according to the invention are aqueous use forms such as emulsion concentrates, suspensions, pastes, wettable powders or water-dispersible granules, which can be prepared, for example, by adding water. To prepare emulsions, pastes or oil dispersions, the substances and/or compositions, what are known as the substrates, as such or dissolved in an oil or solvent, may be homogenized in water by means of wetter, adhesive, dispersant or emulsifier. However, concentrates composed of active substance, wetter, adhesive, dispersant or emulsifier and, if appropriate, solvent or oil may be prepared, and these concentrates are suitable for dilution with water.

Suitable surface-active substances are the alkali metal salts, alkaline earth metal salts and ammonium salts of aromatic sulfonic acids, for example lignosulfonic acid, phenolsulfonic acid, naphthalenesulfonic acid and dibutylnaphthalenesulfonic acid, and of fatty acids, alkylsulfonates and alkylarylsulfonates, alkylsulfates, lauryl ether sulfates and fatty alcohol sulfates, and salts of sulfated hexa-, hepta- and octadecanols, and of fatty alcohol glycol ether, condensates of sulfonated naphthalene, and its derivatives with formaldehyde, condensates of naphthalene or of the naphthalenesulfonic acids with phenol and formaldehyde, polyoxyethylene octylphenyl ether, ethoxylated isooctylphenol, octylphenol or nonylphenol, alkylphenyl polyglycol ethers, tributylphenyl polyglycol ethers, alkylaryl polyether alcohols, isotridecyl alcohol, fatty alcohol/ethylene oxide condensates, ethoxylated castor oil, polyoxyethylene alkyl ethers or polyoxypropylene alkyl ethers, lauryl alcohol polyglycol ether acetate, sorbitol esters, lignin-sulfite waste liquors or methylcellulose.

Powders, materials for spreading and dusts as solid carriers can be prepared advantageously by mixing or concomitantly grinding the active substances with a solid carrier.

Granules, for example coated granules, impregnated granules and homogeneous granules, can be prepared by binding the active ingredients to solid carriers. Examples of solid carriers are mineral earths such as silicas, silica gels, silicates, talc, kaolin, limestone, lime, chalk, bole, loess, clay, dolomite, diatomaceous earth, calcium sulfate, magnesium sulfate, magnesium oxide, ground synthetic materials, fertilizers such as ammonium sulfate, ammonium phosphate, ammonium nitrate, ureas and products of vegetable origin such as cereal meal, tree bark meal, wood meal and nutshell meal, cellulose powders or other solid carriers.

The concentrations of the substances and/or compositions according to the invention in the ready-to-use preparations can be varied within wide ranges. In general, the formulations comprise 0.001 to 98% by weight, preferably 0.01 to 95% by weight, of at least one active ingredient. In this context, the active ingredients are employed in a purity of 90% to 100%, preferably 95% to 100% (according to NMR spectrum).

The herbicidal compositions or the substances can be applied pre- or post-emergence. If the active ingredients are less well tolerated by specific crop plants, application techniques may be used in which the herbicidal compositions or substances are sprayed, with the aid of the spraying apparatus, in such a way that coming into contact with the leaves of the sensitive crop plants is avoided as far as possible, while the active ingredients reach the leaves of undesired plants which grow underneath, or the bare soil surface (post-directed, lay-by).

To widen the spectrum of action and to achieve synergistic effects, the substances and/or compositions according to the invention may be mixed with a large number of representatives of other groups of herbicidal or growth-regulatory active ingredients and applied concomitantly. Suitable examples of components in mixtures are 1,2,4-thiadiazoles, 1,3,4-thiadiazoles, amides, aminophosphoric acid and its derivatives, aminotriazoles, anilides, (het)-aryloxyalkanoic acids and their derivatives, benzoic acid and its derivatives, benzothiadiazinones, 2-aroyl-1,3-cyclohexanediones, hetaryl aryl ketones, benzylisoxazolidinones, meta-CF₃-phenyl derivatives, carbamates, quinolinecarboxylic acid and its derivatives, chloroacetanilides, cyclohexane-1,3-dione derivatives, diazines, dichloropropionic acid and its derivatives, dihydrobenzofurans, dihydrofuran-3-ones, dinitroanilines, dinitrophenols, diphenyl ethers, dipyridyls, halocarboxylic acids and their derivatives, ureas, 3-phenyluracils, imidazoles, imidazolinones, N-phenyl-3,4,5,6-tetrahydrophthalimides, oxadiazoles, oxiranes, phenols, aryloxy- or heteroaryloxyphenoxypropionic esters, phenylacetic acid and its derivatives, phenylpropionic acid and its derivatives, pyrazoles, phenylpyrazoles, pyridazines, pyridinecarboxylic acid and its derivatives, pyrimidyl ethers, sulfonamides, sulfonylureas, triazines, triazinones, triazolinones, triazolecarboxamides, uracils.

Moreover, it may be useful to apply the substances and/or compositions according to the invention, alone or in combination with other herbicides, as a joint mixture together with other crop protection agents, for example with agents for controlling pests or phytopathogenic fungi or bacteria. Also of interest is the miscibility with mineral salt solutions which are employed for alleviating nutritional and trace element deficiencies. Nonphytotoxic oils and oil concentrates may also be added.

Depending on the intended aim of the control measures, the season, the target plants and the growth stage, the application rate of active ingredient (=substance and/or composition) is from 0.001 to 3.0, preferably 0.01 to 1.0, kg of active substance per ha.

The invention furthermore relates to the use of a substance identified by one of the methods or compositions according to the invention comprising these substances for use as a herbicide or for regulating the growth of plants.

Moreover, the invention relates to a kit encompassing the nucleic acid construct according to the invention, the substances according to the invention, for example the antibody according to the invention, the antisense nucleic acid molecule according to the invention and/or an antagonist and/or a herbicidal substance identified in accordance with the methods according to the invention, and the composition described hereinbelow.

The invention furthermore relates to a composition comprising the substance according to the invention, the antibody according to the invention, the antisense nucleic acid construct according to the invention and/or an antagonist according to the invention and/or a substance according to the invention identified by a method according to the invention.

The invention is illustrated in greater detail by the examples which follow, which should not be taken as limiting.

### EXAMPLES

a) Molecular-Biological Methods

Molecular-biological methods as employed herein are those of the prior art and are described in various references such as, for example, Sambrook et al., Molecular Cloning, eds., Cold Spring Harbor Laboratory Press, Cold Spring Harbor, N.Y. (1989), Reiter et al., Methods in Arabidopsis Research, World Scientific Press (1992), Schultz et al., Plant Molecular Biology Manual, Kluwer Academic Publishers (1998) and Martinez-Zapater and Salinas, Methods in Molecular Biology, Vol. 82: Arabidopsis Protocols eds., Humana Press Inc., Totowa, N.J. These references describe the customary standard methods for the production, identification and cloning of mutants caused by T-DNA insertions. In addition, a further customary method for the identification of insertion sites as was described, for example, by Spertini et al., Biotechniques 27: 308-314 (1999), was resorted to. The sequencing was carried out by AGOWA (Berlin).

b) Materials

Unless otherwise specified in the text, the chemicals used were obtained in analytical-grade quality from Fluka (Neu-Ulm), Merck (Darmstadt), Roth (Karlsruhe), Serva (Heidelberg) and Sigma (Deideshofen). Solutions were prepared using pure, pyrogen-free water, referred to in the text as 1110, obtained from an ion-exchange system by TKA (Niederelbert). Restriction nucleases, DNA-modifying enzymes and molecular biology kits and oligonucleotides were obtained from Amersham Pharmacia (Freiburg), Biometra (Göttingen), Dynal (Hamburg), Gibco-BRL (Gaithersburg, Md., USA), Invitrogen (Groningen, Netherlands), MBI Fermentas (St. Leon Rot), New England Biolabs (Schwalbach, Taunus), Novagen (Madison, Wisc., USA), Qiagen (Hilden), Roche Diagnostics (Mannheim), Stratagene (Amsterdam, Netherlands), TTB-Molbiol (Berlin). Unless otherwise specified, the products were employed in accordance with the manufacturers' instructions.

c) Generation and Identification of Lines Which Segregate for Lethal Mutations

Wild-type Arabidopsis plants of ecotype C24 were transformed by means of a modified “in planta” transformation protocol (Bechthold et al., 1992; Clough and Bent, 1998), and transgenic F₁ plants were selected by means of antibiotic or herbicide resistances (inter alia Clearfield). T₂ seeds of these lines were placed on sterile medium and on compost and, after 7 days' growth under standard conditions, inspected visually for the occurrence of dying seedlings. Features which were observed in particular were changes in pigmentation down to its complete absence, and morphological anomalies. Only those lines for which a segregation ratio of approx. 2:1-3:1, that is to say twice to three times the amount of resistant plants to sensitive plants, was determined in a parallel study were studied further. This ratio indicates a single integration site which causes the resistance.

Various lines which segregate for a lethal mutation were found. The molecular-biological analyses were carried out as described for Examples 1 to 4. From Example 7, the SEQ ID NO. which describes each of the mutated sequences for the respective lines is stated.

### Example 1

### Identification and Analysis of Line P9, Which Segregates for a Lethal Mutation

Line 9 (see SEQ ID NO: 3) was identified as described above as a line which segregates for a mutation which is lethal for the seedling. The accurate determination of the segregation revealed that 25% of the progeny showed the albino phenotype, 25% of the progeny sensitivity to the selection and 50% of the progeny resistance to the selection. This segregation ratio is expected when exclusively the homozygously-resistant seedlings are homozygous for the mutation and thus show the recessive phenotype, which is why the T-DNA insertion is coupled very closely to the lethal mutation. The coupling was furthermore checked in a cosegregation analysis. To this end, the progeny of 34 resistant wild-type plants of line P9 was analyzed. Again, albinos were found in the progeny in all cases. This fact allows the conclusion that the resistance-mediating T-DNA insertion and the mutation are always inherited together and therefore coincide (with a high degree of probability). Thereupon, the line was studied in terms of molecular biology in order to accurately determine the site of the T-DNA integration. To this end, the genomic DNA was isolated from approximately 50 mg of tissue of these plants using standard products and methods (columns from Qiagen, Hilden, Germany, or Phytopure kit from Amersham Pharmacia, Freiburg, Germany) and studied for integrity and quantity by separation by means of gel electrophoresis. The amplification of the genomic sequences adjacent to the T-DNA was carried out by means of a modified adaptor PCR protocol (Spertini et al., 1999). For each combined restriction and adaptor ligation, approximately 50 to 100 ng of the DNA were cleaved with the restriction enzymes BglII, MunI, Spel, PspI406I/BspI991, AflII and the adaptor consisting of the annealed oligos 5′CTAATACGACTCACTATAGGGCTCGAGCGGCCGGGCAGGT-3′ and 5′NN₍₂₋₄₎ACCTGCCCAA-3′, where 5′NN₍₂₋₄₎ represent the overhang matching the enzyme in question. One μl of this genomic DNA provided with adaptors was employed for an amplification of the sequences flanking the T-DNAs, using an adaptor-specific primer (5′GGATCCTAATACGACTCACTATAGGGC-3′) and a gene-specific primer (LB1: 5′TGACGCCATTTCGCCTTTTCA-3′ for the left border and RB 1: 5′CAACTTAATCGCCTTGCAGCACA-3′ for the right border). The PCR was carried out under standard conditions for 7 cycles at an annealing temperature of 72° C. and for 32 cycles at an annealing temperature of 65° C. in a reaction volume of 25 μl. The amplificate obtained was diluted 1:50 in H₂O, and one μl of this dilution was employed for a second round of amplification (5 cycles at an annealing temperature of 67° C. and 28 cycles at an annealing temperature of 60° C., otherwise standard PCR conditions). To this end, “nested” primers, i.e. primers which are positioned further inside on the PCR product (5′-TATAGGGCTCGAGCGGC-3′ for the adaptor, LB2: 5′CAGAAATGGATAAATAGCCTTGCTTCC-3′ for the left border and RH2: 5′AGCTGGCGTAATAGCGAAGAG-3′ for the right border) were employed, thus increasing the specificity and selectivity of the amplification. An aliquot of the amplificate, which was obtained in a reaction volume of 50 μl, was subjected to analysis by gel electrophoresis. For line P9, a 1350 bp fragment was identified for the left T-DNA border and a 750 bp fragment for the right border for the enzyme combination Psp14O6I/BspII9I. The products were sequenced by means of the primers RBseq and Lbseq (5′CAATACATTACACTAGCATCTG-3′). Both products identified the identical region in the Arabidopsis genome. The successful identification was verified by a PCR reaction with a primer specific for the derived flanking sequence and a primer specific for the T-DNA, RB1. Obtaining the PCR product of the predicted size which is specific for this line confirmed the successful identification of the insertion site of the T-DNA. BLAST analysis of the isolated sequences (BLASTN, Altschul et al., 1990) J Mol. Biol. 215:403-410) demonstrated the insertion of the left border in position 53498 and of the right border in position 54283 of clone MVII1 of chromosome III (EMBL|AP000419). An ORF (MVI11.13) which encodes a “DNA repair protein RAD-54-like” protein is annotated in this region. This ORF, which is interrupted by the insertion, shows a high degree of homology with the yeast DNA repair protein RHP54 (Muris et al., J. Cell. Sei., 1996) and similarities with a series of other DNA-binding proteins.

### Example 2

### Identification and Analysis of Line P38, Which Segregates for a Lethal Mutation

Line P38 was identified as described above as a line which segregates for a mutation which is lethal to the seedling. The accurate determination of the segregation revealed that 25% of the progeny showed the albino phenotype, 25% of the progeny sensitivity to the selection and 50% of the progeny resistance to the selection. This segregation ratio is expected when exclusively the homozygously-resistant seedlings show the phenotype and the T-DNA insertion is therefore coupled very closely to the lethal mutation. The coupling is to be tested further using cosegregation analysis. To this end, the progeny of 34 resistant wild-type plants of line P38 was analyzed. Again, albinos were found in the progeny in all cases. This fact allows the conclusion that the resistance-mediating T-DNA insertion and the mutation are always inherited together and therefore coincide (with a high degree of probability). The molecular-biological analysis was carried out as described in Example 1. For line P38, a 350 bp fragment was identified for the left T-DNA border in the case of the enzyme MunI. The product was sequenced by means of the primer Lbseq and showed the expected identity with a region of the Arabidopsis genome. The successful identification was verified by a PCR reaction with a primer specific for the derived flanking sequence and a primer specific for the T-DNA, LB1. Obtaining the PCR product of the predicted size which is specific for this line confirmed the successful identification of the insertion site of the T-DNA. BLAST analysis of the isolated sequence (BLASTN, Altschul et al., 1990, J Mol. Biol. 215:403-410) demonstrated the insertion of the T-DNA in position 42163 of the BAC clone F3E22 of chromosome III (EMBLNEW|AC023912). According to the annotation of this region, integration took place into a predicted ORF (F3E22.13), which showed significant similarity with various thioredoxins. Then, primers were synthesized for the predicted 5′ and 3′ end and employed for a standard PCR with Arabidopsis cDNA. The mRNA was isolated from the seedlings by means of oligo-dT Dynabeads from Dynal, and the cDNA was generated therefrom using a cDNA synthesis kit from Gibco-BRL and an oligo dT primer. The PCR product obtained was ligated into a TA vector (pCRScriptII, Invitrogen) and sequenced. The sequence showed complete agreement with the predicted sequence and gene structure for this ORF. Accordingly, the inventors herewith document for the first time the experimental confirmation of the existence of this ORF in the predicted structure.

### Example 3

### Identification and Analysis of Line P44, Which Segregates for a Lethal Mutation

Line P44 was identified as described above as a line which segregates for a mutation which is lethal to the seedling. The accurate determination of the segregation revealed that 25% of the progeny showed the albino phenotype, 25% of the progeny sensitivity to the selection and 50% of the progeny resistance to the selection. This segregation ratio is expected when exclusively the homozygously-resistant seedlings show the phenotype and the T-DNA insertion is therefore coupled very closely to the lethal mutation. The coupling was furthermore tested in a cosegregation analysis. To this end, the progeny of 34 resistant wild-type plants of line P44 was analyzed. Again, albinos were found in the progeny in all cases. This fact allows the conclusion that the resistance-mediating T-DNA insertion and the mutation are always inherited together and therefore coincide (with a high degree of probability). The molecular-biological analysis was carried out as described in Example 1. For line P44, a 350 bp fragment was identified for the enzyme MunI and a 500 bp fragment for the enzyme BglII, in each case for the left T-DNA border. These products were sequenced by means of the primer Lbseq and defined the identical position in the Arabidopsis genome. The successful identification was verified by a PCR reaction with a primer specific for the derived flanking sequence and a primer specific for the T-DNA, LB1. Obtaining the PCR product of the predicted size which is specific for this line confirmed the successful identification of the insertion site of the T-DNA. BLAST analysis of the isolated sequence (BLASTN, Altschul et al., 1990, J Mol. Biol. 215:403-410) demonstrated the insertion of the T-DNA in position 66762 of the TAC clone K15C23 (EMBLALERT|AB024024). According to the annotation of this region, integration took place in a predicted ORF (K15C23.10), whose derived amino acid sequence shows no significant homologies. In contrast, weak homologies are found with mouse (Accession Q60992) and human (Accession: P52735) VAV2 proteins. Then, primers were synthesized for the predicted 5′ and 3′ end of the ORF and employed for a standard PCR with Arabidopsis cDNA. cDNA was generated as described in Example 3. The sequence showed complete agreement with the predicted sequence and gene structure for this ORF. Accordingly, the inventors herewith document for the first time the experimental confirmation of the existence of this ORF in the predicted structure.

### Example 4

### Identification and Analysis of Line P77, Which Segregates for a Lethal Mutation

Line P77 was identified as described above as a line which segregates for a mutation which is lethal to the seedling. The accurate determination of the segregation revealed that 25% of the progeny showed the albino phenotype, 25% of the progeny sensitivity to the selection and 50% of the progeny resistance to the selection. This segregation ratio is expected when exclusively the homozygously-resistant seedlings show the phenotype and the T-DNA insertion is therefore coupled very closely to the lethal mutation. The coupling was furthermore tested in a cosegregation analysis. To this end, the progeny of 34 resistant wild-type plants of line P77 was analyzed. Again, albinos were found in the progeny in all cases. This fact allows the conclusion that the resistance-mediating T-DNA insertion and the mutation are always inherited together and therefore coincide (with a high degree of probability). The molecular-biological analysis was carried out as described in Example 1. For line P77, a 650 bp fragment was identified for the left T-DNA border for the enzyme combination Psp14061/Bsp1191. The products were sequenced by means of the primer Lbseq. The successful identification was verified by a PCR reaction with a primer specific for the derived flanking sequence and a primer specific for the T-DNA, LB1. Obtaining the PCR product of the predicted size which is specific for this line confirmed the successful identification of the insertion site of the T-DNA. BLAST analysis of the isolated sequence (BLASTN, Altschul et al., 1990, J Mol. Biol. 215:403-410) showed an absolute agreement of the sequence with a segment on the Arabidopsis chromosome III and demonstrated the insertion of the T-DNA in position 13436 of the BAC clone F24B22 (EMBLATF24B22) and interrupts a predicted open reading frame (F24B22.50) which encodes a protein with a high degree of similarity with various fructokinases, for example from potato (Solanum tuberosum, Accession: P37829) or from the bacterium Vibrio alginolyticus (Accession: P22824). Then, primers were synthesized for the predicted 5′ and 3′ end of the ORFs and employed for a standard PCR with Arabidopsis cDNA. cDNA was generated as described in Example 3. The sequence showed complete agreement with the predicted sequence and gene structure for this ORF. Accordingly, the inventors herewith document for the first time the experimental confirmation of the existence of this ORF in the predicted structure.

### Example 5

### Identification and Analysis of Lines P95, P98, P99b, P102 and P103, Which Segregate for a Lethal Mutation

Analogously to the abovementioned Examples 1 to 4, clones P95, P98, P99b, P102 and P103 were identified as lines which segregate for mutations which are lethal for the seedling. The molecular-biological procedures or analyses were carried out as described in Examples 1 to 4.

In line P95, the T-DNA is inserted in position 35442 of BACT5L19 (Accession number AL049481) of the Arabidopsis chromosome IV. In line P98, the T-DNA was inserted in position 54861 of the P1 clone MVA3 (Accession number: AB006706) of chromosome V. In line P99b, the insertion of the T-DNA was found to be in position 66042 of the BAC F10M10 (AL035521) on chromosome IV. In clone P102, the insertion of the T-DNA was found to be on chromosome IV in the region of the contig fragment 69 in position 46342-46355 (AL 161573). Line P103 showed an insertion of the T-DNA in position 57314 of the BAC F11F8 (AC016661) of chromosome I.

### Example 6

### Identification and Analysis of Lines P91 and P99a, Which Segregate for a Lethal Mutation

Analogously to the abovementioned examples, clones P91 and P99a were identified as lines which segregate for mutations which are lethal for the seedling. The molecular-biological analyses were carried out as described for Examples 1 to 4.

### Example 7

Line A300364 (Seq ID No.: 26 [nucleic acid] and 27 [protein]) was identified analogously to the abovementioned examples. Line A300364 segregates for an embryo-lethal mutation. A 2:1 segregation was observed. When 35 lines were studied, an absolute cosegregation was observed between the T-DNA and the mutation which leads to the albino phenotype. In these lines, the T-DNA is inserted in position 39517 of chromosome II (EMBL|AC004238). In this position, the insertion interrupts, and thus inactivates, an ORF (At2g34860) which encodes an unknown protein (AAC12826.1). In Blastp comparisons with standard settings, the protein shows homology over a region of 40 amino acids with various DNAJ chaperone proteins (heat-shock protein 40) or the DNAJ protein (Q9UXR9) from Methanosarcina thermophila (Hoffmann-Bang et al., Gene 238 (2), 387-395 (1999)).

### Example 8

Line A301034 (Seq ID No.: 28 [nucleic acid] and 29 [protein]) was identified analogously to the abovementioned examples. Line A301034 segregates for an embryo-lethal mutation. A 2:1 segregation was observed. When 35 lines were studied, an absolute cosegregation was observed between the T-DNA and the mutation which leads to the albino phenotype. The T-DNA is inserted on chromosome V in position 25928 of the BAC T21H19 (EMBL|ATT21H19). In this position, the insertion interrupts a gene (T21H19_{—}100) which encodes a putative protein (CAC01859.1). This shows homology with other putatitve Arabidopsis proteins. The derived protein sequence shows marked homologies with the maize CRS1 gene product (AAG00595), which is required for splicing the group II intron of the chloroplast gene atpF. Seq ID No.: 43 shows the genomic sequence of line A301034 from the start codon to the stop codon, including introns.

The activity can be tested, for example, in assays as they are described in Bock, Nucleic Acids Res., 1995, 23, 2544-7.

### Example 9

Line A300377 (Seq ID No.: 30 [nucleic acid] and 31 [protein]) was identified analogously to the abovementioned examples. Line A300377 segregates for an embryo-lethal mutation. A 2:1 segregation was observed. When 35 lines were studied, an absolute cosegregation was observed between the T-DNA and the mutation which leads to the albino phenotype. The T-DNA is inserted in position 14509 of the P1 clone MRN17 (AB005243) and thus very likely in the 3′-untranslated region of an alanyl-tRNA synthetase (BAB10601.1).

### Example 10

Line A300841 (Seq ID No.: 32 [nucleic acid] and 33 [protein]) was identified analogously to the abovementioned examples as being essential. Line A300841 segregates for an embryo-lethal mutation. A 2:1 segregation was observed. When 35 lines were studied, an absolute cosegregation was observed between the T-DNA and the mutation which leads to the albino phenotype. In this line, the T-DNA is inserted in position 3183 of the BAC T14P8, which corresponds to position 143432 in the contig fragment 6 of chromosome IV. Owing to the insertion in this position, an open reading frame for a putatitive chloroplast outer envelope 86-like protein is destroyed. ESTs for this ORF (CAB80744.1) already exist in databases (EST gb:AI998804.1, R90258, AA651438). The protein encoded shows a high degree of homology with the chloroplast outer envelope 86 protein OEP86 from pea, P. sativum, GenBank Accession Number Z31581 and has an ATP/GTP binding motif (P-loop).

The activity of an OEP86 can be tested in assays, for example as described or cited in Muckel, J. Biol. Chem., 1996, 271, 23846-52, Young, Plant Physiol., 1999, 121, 237-44 or in the review Keegstra, Curr. Opin. Plant Biol., 1999, 2, 471-6.k

### Example 11

Line 2266c (Seq ID No.: 34 [nucleic acid] and 35 [protein]) was identified analogously to the abovementioned examples. Line 2266c segregates for an embryo-lethal mutation. A 2:1 segregation was observed. The T-DNA is inserted in position 26501 of the BAC F6N18 (AC017118) of chromosome I. In this position, the insertion interrupts an ORF whose derived amino acid sequence (AAF25967.1) shows marked similarity to an Arabidopsis FMRF amide propeptide isolog (gi|1871179).

### Example 12

Line P61 (Seq ID No.: 36 [nucleic acid] and 37 [protein]) was identified analogously to the abovementioned examples. Line P61 segregates for an embryo-lethal mutation. A 2:1 segregation was observed. When 35 lines were studied, an absolute cosegregation was observed between the T-DNA and the mutation which leads to the albino phenotype. The T-DNA is inserted in position 28640 of the BAC F4B12 (EMBLNEW|AP001299) on chromosome III. The insertion prevents the expression of an ORF which starts in position 28705 and which encodes an unknown protein (BAB02572.1) with a low degree of homology with proteosomal protein 26S PROTEASOME SUBUNIT S5B, (Deveraux, Q., Jensen, C. and Rechsteiner, M., Molecular cloning and expression of a 26 S protease subunit enriched in dileucine repeats, J. Biol. Chem. 270 (40), 23726-23729 (1995)).

### Example 13

Line A300857 (Seq ID No.: 38 [nucleic acid] and 39 [protein]) was identified analogously to the abovementioned examples. Line A300857 segregates for an embryo-lethal mutation. When 35 lines were studied, an absolute cosegregation was observed between the T-DNA and the mutation which leads to the albino phenotype. In this line, the T-DNA is inserted in position 51122 of the BAC T10024 of chromosome I (EMBL:AC007067). In this position, the insertion interrupts, and thus inactivates, an ORF (T10O24.14) which encodes an unknown protein (AAD39574.1). Seq. ID. No. 42 shows the corresponding genomic sequence.

### Example 14

Line A300367 (Seq ID No.: 40 [nucleic acid] and 41 [protein]) was identified analogously to the abovementioned examples. Line A300367 segregates for an embryo-lethal mutation. When 35 lines were studied, an absolute cosegregation was observed between the T-DNA and the mutation which leads to the albino phenotype. In this line, the T-DNA is inserted in position 31058 “contig fragments” 86 (EMBL:ATCHRIV86) of chromosome IV. Owing to the insertion of few base pairs upstream of the start codon (51073) of a geranylgeranyl-pyrophosphate synthase (Bartley and Scolnik, 1994) (Plant Physiol. 104, 1469-1470, 1994), Accession L25813, it is highly probable that the functionality of the gene is destroyed by interfering with transcription, transcript stability or at least translation. Thus, Okada, Plant Physiol., 2000, 122, 1045-56, describes five different GGPPs in Arabidopsis which are localized in three different compartments. Surprisingly, the protein or transcript, probably a GGPP, shown herein is essential.

The activity of a geranylgeranyl-pyrophosphate synthase can be tested in test systems, for example as described in Zhu et al., Plant Cell Physiol., 1997, 38, 337-61, or Okada, Plant Physiol., 2000, 122, 1045-56.

### Example 15

Line 305735 (SEQ ID NO: 44 [nucleic acid] and SEQ ID NO: 45 [protein encoded by the above nucleic acid]) was identified analogously to the abovementioned examples. Line 305735 segregates for an albino-lethal mutation, which cosegregates with the resistance marker and thus with the T-DNA. The T-DNA is integrated in position 46571 of the sequence ATCHRIV69, Accession number AL161573. The insertion of this position interrupts the ORF AT4g28590, which encodes a hypothetical protein which has a “cecropin” family signature (AA237-245).

### Example 16

Line 303726 (SEQ ID NO: 46 [nucleic acid] and SEQ ID NO: 47 [protein encoded by the above nucleic acid]) was identified analogously to the abovementioned examples. Line 303726 segregates for an albino-lethal mutation which cosegregates with the resistance marker and thus with the T-DNA. The T-DNA is inserted in position 51568 of the BAC AC004669 on chromsome 2. Owing to the insertion at this position, it is highly probable that the transcription and thus the function of the ORF At2g30950 is prevented or adversely affected. This ORF encodes a putative ftsH chloroplast protease.

### Example 17

Line 304249 (SEQ ID NO: 48 [nucleic acid] and SEQ ID NO: 49 [protein encoded by the above nucleic acid]) was identified analogously to the abovementioned examples. Line 304249 segregates for an albino-lethal mutation which cosegregates with the resistance marker and thus with the T-DNA. The T-DNA is inserted in position 13004 of the BAC ATF19B15, Accession number AL078470 destroys the ORF F19B15.40 which encodes the Arabidopsis “AIM1” protein (CAB43915.1). A plurality of ESTs, GB:Z31666, gb:Z33957, Z31666, have already been described for this ORF. This protein is a peroxysomal tetrafunctional enzyme of the fatty acid metabolism.

### Example 18

Line 304264 (SEQ ID NO: 50 [nucleic acid] and SEQ ID NO: 51 [protein encoded by the above nucleic acid]) was identified analogously to the abovementioned examples. Line 304264 segregates for an albino-lethal mutation, which cosegregates with the resistance marker and thus with the T-DNA. The T-DNA is inserted in position 63762 of the BAC AB020742. The insertion site is located approximately 240 base pairs upstream of the start codon for an ORF K21H1.19, which encodes a UDP-glucuronyl transferase-like protein. Owing to the insertion of the T-DNA in this position, it is highly probable that the transcription is modified or prevented and the function of the gene thus destroyed.

### Example 19

Line 304485 (SEQ ID NO: 52 [nucleic acid] and SEQ ID NO: 53 [protein encoded by the above nucleic acid]) was identified analogously to the abovementioned examples. Line 304485 segregates for an albino-lethal mutation, which cosegregates with the resistance marker and thus with the T-DNA. The T-DNA is inserted in position 25034 of the BAC AC006438 on chromosome 2. Owing to the integration at this site, the T-DNA destroys the ORF At2g15820 which encodes an unknown protein.

### Example 20

Line 304652 (SEQ ID NO: 54 [nucleic acid) and SEQ ID NO: 55 [protein encoded by the above nucleic acid]) and SEQ ID NO: 56 [nucleic acid] and SEQ ID NO: 57 [protein encoded by the above nucleic acid] was identified analogously to the abovementioned examples. Line 304652 segregates for an albino-lethal mutation, which cosegregates with the resistance marker and thus with the T-DNA. The T-DNA is inserted in position 6309 of the BAC ATF12B17, Accession AL353995, on chromosome 5. Two different open reading frames are annotated for the adjacent region. Owing to the integration of approx. 382 base pairs upstream of the ORFs ATF12B17_{—}20 and ATF12B17_{—}10, it is highly probable that the transcription and the functionality of the genes, which encode an FPF1-like (flowering promoting factor 1) protein (ATF12B17_{—}20, SEQ ID NO: 54 and SEQ ID NO: 55) and a protein (ATF12B17_{—}10, SEQ ID NO: 56 and SEQ ID NO: 57) with similarity to the Homo sapiens KIAA1038 protein, is disrupted or prevented.

### Example 21

Line 304656 (SEQ ID NO: 58 [nucleic acid] and SEQ ID NO: 59 [protein encoded by the above nucleic acid]) was identified analogously to the abovementioned examples. Line 304656 segregates for an albino-lethal mutation, which cosegregates with the resistance marker and thus with the T-DNA. The T-DNA is inserted in position 35169 of the BAC F24P17 (Accession AC011623) on chromosome 3. In this position, the insertion interrupts, and thus destroys, an ORF 24P17.10, which encodes an unknown protein. The blastp alignment with standard settings reveals pronounced homologies with a nodulin/glutamate-ammonia-ligase-like protein.

### Example 22

Line 302192 (SEQ ID NO: 60 [nucleic acid] and SEQ ID NO: 61 [protein encoded by the above nucleic acid]) was identified analogously to the abovementioned examples. Line 302192 segregates for an albino-lethal mutation, which cosegregates with the resistance marker and thus with the T-DNA. The T-DNA is inserted in position 43178 of the BAC AB022211 on chromosome 5. Owing to the integration of approx. 454 base pairs upstream of the ORF K1L20.13, it is highly probable that the transcription, and the thus the function, of the gene which encodes an SHI-like zinc finger protein (short internodes) is disrupted or prevented.

### Example 23

Line 302636 (SEQ ID NO: 62 [nucleic acid] and SEQ ID NO: 63 [protein encoded by the above nucleic acid]) was identified analogously to the abovementioned examples. Line 302636 segregates for an albino-lethal mutation, which cosegregates with the resistance marker and thus with the T-DNA. The T-DNA is inserted in position 141376 of the BAC ATF4P12 (Accession: AL132966) on chromosome 3. The integration of the T-DNA at this position interrupts, and thus inactivates, the ORF F4P12_{—}400, which encodes a protein with similarity to crp1 from Zea mays, PIR:T01685. Moreover, this ORF contains the ESTs gb:AI999771.1, T45254, AA713158″.

### Example 24

Line 302894 (SEQ ID NO: 64 [nucleic acid] and SEQ ID NO: 65 [protein encoded by the above nucleic acid]) was identified analogously to the abovementioned examples. Line 302894 segregates for an albino-lethal mutation, which cosegregates with the resistance marker and thus with the T-DNA. The T-DNA is inserted in position 23970 of the BAC ATT21H19 (Accession: AL391148) on chromosome 5. The insertion of the T-DNA at this position interrupts an ORF (T21H19_{—}100), which encodes a putative protein with similarities with hypothetical proteins from Arabidopsis. Moroever, the blastp analysis reveals a pronounced homology with CRS1 from Zea mays Accession AAG00595, which is a group II intron splicing factor (Till,B et al., RNA 7 (9), 1227-1238 (2001)).

### Example 25

Line 305146 (SEQ ID NO: 66 [nucleic acid] and SEQ ID NO: 67 [protein encoded by the above nucleic acid]) was identified analogously to the abovementioned examples. Line 305146 segregates for an albino-lethal mutation, which cosegregates with the resistance marker and thus with the T-DNA. The T-DNA is inserted in position 65706 of the P1 clone MOP9 (Accession: AB006701) on chromosome 5. The insertion of the T-DNA at this position interrupts the ORF of the gene At5g24315, which encodes an unknown protein.

### Example 26

Acc.: AL163816 Name: ATT20O10

Line 305156 (SEQ ID NO: 68 [nucleic acid] and SEQ ID NO: 69 [protein encoded by the above nucleic acid]) was identified analogously to the abovementioned examples. Line 305156 segregates for an albino-lethal mutation, which cosegregates with the resistance marker and thus with the T-DNA. The T-DNA is inserted in position 898 of the BAC ATT20O10 MOP9 (Accession: AL163816) on chromosome 3. The insertion of the T-DNA at this position interrupts an ORF (T20O10_{—}10), which encodes a protein with a high degree of similarity to the Synechocystis translation releasing factor RF-1 (PIR:S76914). The derived amino acid sequence contains a prokaryotic type class I peptide chain detachment factor motif, AA280-296.

### Example 27

Line 304044 (SEQ ID NO: 70 [nucleic acid] and SEQ ID NO: 71 [protein encoded by the above nucleic acid]) was identified analogously to the abovementioned examples. Line 304044 segregates for an albino-lethal mutation, which cosegregates with the resistance marker and thus with the T-DNA. The T-DNA is inserted in position 44121 of the BAC ATAP22 MOP9 (Accession: Z99708) on chromosome 4. The insertion of the T-DNA at this position interrupts an ORF (C7A10.610), which encodes a protein with a high degree of similarity with an allergen (“minor allergen”) from Alternaria alternata (PIR2:S43111). Moreover, the ESTs gb:R64949, AA651052 have already been found for this ORF.

Acc.: Z99708 Name: ATAP22

### Example 28

Line 140412 (SEQ ID NO: 72 [nucleic acid] and SEQ ID NO: 73 [protein encoded by the above nucleic acid]) was identified analogously to the abovementioned examples. Line 140412 segregates for an albino-lethal mutation, which cosegregates with the resistance marker and thus with the T-DNA. The T-DNA is inserted in position 68520 of the sequence AC006264 on chromosome 2. The insertion of the T-DNA interrupts the 3′UTR of the gene At2g21160, and it is therefore highly probable that the function of the ORF is prevented owing to the destabilization of the transcript. The ORF At2g21160 encodes the alpha-subunit of a putative signal sequence receptor.

Acc: AC006264 Name: AC006264

### Example 29

Line 159012 (SEQ ID NO: 74 (nucleic acid] and SEQ ID NO: 75 [protein encoded by the above nucleic acid]) was identified analogously to the abovementioned examples. Line 159012 segregates for an albino-lethal mutation, which cosegregates with the resistance marker and thus with the T-DNA. The T-DNA is inserted in position 127261 of the sequence ATCHRIV3 (fragment No. 3), Accession AL161491 on chromosome 4. The insertion of the T-DNA interrupts the ORF AT4g01220, which contains the ESTs gb:AA597894, AA597304 and encodes unknown protein.

Acc: AL161491 Name: ATCHRIV3 (fragment No. 3)

### Example 30

Line 106037 (SEQ ID NO: 76 [nucleic acid] and SEQ ID NO: 77 [protein encoded by the above nucleic acid]) was identified analogously to the abovementioned examples. Line 106037 segregates for an albino-lethal mutation, which cosegregates with the resistance marker and thus with the T-DNA. The T-DNA is inserted in position 50359 of the sequence AC006193 on chromosome 1. The insertion of the T-DNA in this position interrupts the ORF F13O11.11, which encodes an unknown protein. The Blastp analysis with standard settings reveals a similarity with oxidoreductases.

Acc: AC006193 Name: AC006193

### Example 31

Line 126905 (SEQ ID NO: 78 [nucleic acid] and SEQ ID NO: 79 [protein encoded by the above nucleic acid]) was identified analogously to the abovementioned examples. Line 126905 segregates for an embryo-lethal mutation, which cosegregates with the resistance marker and thus with the T-DNA. The T-DNA is inserted in position 71928 of the BAC ATF25L23, Accession AL356014, on chromosome 3. The insertion of the T-DNA in this position interrupts the ORF F25L23_{—}240″, which encodes a farnesyl transferase subunit A.

Acc.: AL356014 Name: ATF25L23

### Example 32

Line 127458 (SEQ ID NO: 80 [nucleic acid] und SEQ ID NO: 81 [protein encoded by the above nucleic acid]) was identified analogously to the abovementioned examples. Line 127458 segregates for an embryo-lethal mutation, which cosegregates with the resistance marker and thus with the T-DNA. The T-DNA is inserted in position 45352 of the BACs T19K24, Accession AC002342, on chromosome 5. The insertion of the T-DNA in this position interrupts the ORF T19K24.18, which encodes the ATP-independent copper transporter RAN1.

Acc.: AC002342 Name: ATAC002342

### Example 33

Line 304249b (SEQ ID NO: 82 [nucleic acid] and SEQ ID NO: 83 [protein encoded by the above nucleic acid]) was identified analogously to the abovementioned examples. Line 304249b segregates for an albino-lethal mutation, which cosegregates with the resistance marker and thus with the T-DNA. The insertion in position 17105 of the BAC ATF19B15, Accession AL078470, destoys the ORF F19B15.50, which shows similarities with glycine-rich proteins and contains the ESTs gb:Z29181, T42831, Z34138, Z33797, Z30844.

### Example 34

Line 304264b (SEQ ID NO: 84 [nucleic acid] and SEQ ID NO: 85 [protein encoded by the above nucleic acid]) was identified analogously to the abovementioned examples. Line 304264b segregates for an albino-lethal mutation, which cosegregates with the resistance marker and thus with the T-DNA. The T-DNA is inserted in position 63762 of the BAC AB020742. The incorporation site is located approximately 340 base pairs upstream of the start codon for an ORF K21H1.18, which has similarity with unknown proteins.

### Example 35

Line 192813 (SEQ ID NO: 86 [nucleic acid] and SEQ ID NO: 87 [protein encoded by the above nucleic acid]) was identified analogously to the abovementioned examples. Line 192813 segregates for an albino-lethal mutation, which cosegregates with the resistance marker and thus with the T-DNA. The T-DNA is inserted on chromosome 1 in position 9869 of the BAC F3O9, Accession AC006341. Owing to the insertion of approx. 323 bp upstream of the start codon for an ORF, F3O9.4, for which a plurality of ESTs have already been identified (gb|F15498, gb|H37515, gb|T41906, gb|T22448, gb|W43356, gb|T20739), it is highly probable that the transcription and thus the function of the gene for this ORF are inhibited. The ORF encodes a protein for which the blastp alignment with standard setting reveals high degrees of homology with a variety of syntaxins and syntaxin-like proteins, also those from plants.

### Example 36

Line 203521 (SEQ ID NO: 88 [nucleic acid] and SEQ ID NO: 89 [protein encoded by the above nucleic acid]) was identified analogously to the abovementioned examples. Line 203521 segregates for an albino-lethal mutation, which cosegregates with the resistance marker and thus with the T-DNA. The T-DNA is inserted on chromosome 2, section 179 of 255 Accession AC006533. Owing to the insertion, the ORF AT2g31830, which encodes a putative inositol-polyphosphate 5′-phosphatase, is destroyed.

### Example 37

Line 206462 (SEQ ID NO: 90 [nucleic acid] and SEQ ID NO: 91 [protein encoded by the above nucleic acid]) was identified analogously to the abovementioned examples. Line 206462 segregates for an albino-lethal mutation, which cosegregates with the resistance marker and thus with the T-DNA. The T-DNA is inserted on chromosome 1 in position 53577-53600 of the BAC F24D7, Accession AC011622. Owing to the insertion at this position, the ORF F24D7.13, which encodes a putative UDP—N-acetylmuramoylalanyl-D-glutamate 2,6-diaminopimelate ligase (murE), is destroyed.

### Example 38

Line 216642 (SEQ ID NO: 92 [nucleic acid] and SEQ ID NO: 93 [protein encoded by the above nucleic acid]) was identified analogously to the abovementioned examples. Line 216642 segregates for an albino-lethal mutation, which cosegregates with the resistance marker and thus with the T-DNA. The T-DNA is inserted on chromosome 3 in position 18529 of the P1 clone MRC8, Accession AB020749. Owing to the insertion at this position, the ORF MRC8.5, which encodes a beta-glucosidase, is destroyed.

### Beispiel 39

Line 219902 (SEQ ID NO: 94 [nucleic acid] and SEQ ID NO: 95 [protein encoded by the above nucleic acid]) was identified analogously to the abovementioned examples. Line 219902 segregates for an albino-lethal mutation, which cosegregates with the resistance marker and thus with the T-DNA. The T-DNA is inserted on chromosome 1 in position 7740 of the BAC F15M4, Accession AC012394. Owing to the insertion at this position, the ORF F15M4.1, which encodes a hydroxymethylglutaryl-CoA reductase, is destroyed.

### Example 40

Line 220801 (SEQ ID NO: 96 [nucleic acid] and SEQ ID NO: 97 [protein encoded by the above nucleic acid]) was identified analogously to the abovementioned examples. Line 220801 segregates for an albino-lethal mutation, which cosegregates with the resistance marker and thus with the T-DNA. The T-DNA is inserted on chromosome 5 in position 15447-15472 of the P1 clone MRN17, Accession AB005243. Owing to this insertion approx. 580 bp upstream of the start codon, it is probable that at least the transciption and thus the function of the ORF MRN17.4 is destroyed. This ORF encodes a GDSL-motif-lipase/hydrolase-like protein.

### Example 41

Line 224933 (SEQ ID NO: 98 [nucleic acid] and SEQ ID NO: 99 [protein encoded by the above nucleic acid]) was identified analogously to the abovementioned examples. Line 224933 segregates for an albino-lethal mutation, which cosegregates with the resistance marker and thus with the T-DNA. The T-DNA is inserted on chromosome 4, ESSA I FCA Contig-Fragment No. 3., Accession Z97338, in position 107932-107997. Owing to this insertion, the functionality of the ORF d13705c, which encodes a cellulose-synthase-like protein, is destroyed.

### Example 42

Line 229091 (SEQ ID NO: 100 [nucleic acid] and SEQ ID NO: 101 [protein encoded by the above nucleic acid]) was identified analogously to the abovementioned examples. Line 229091 segregates for an albino-lethal mutation, which cosegregates with the resistance marker and thus with the T-DNA. The T-DNA is inserted on chromosome 5, TAC clone:K5J14, Accession AB023032, in position 55778. Owing to the insertion at this position, the functionality of the ORF K5J14.11, which encodes a maize-crp1-protein-like protein, is destroyed.

### Example 43

Line 246473 (SEQ ID NO: 102 [nucleic acid] and SEQ ID NO: 103 [protein encoded by the above nucleic acid]) was identified analogously to the abovementioned examples. Line 246473 segregates for an albino-lethal mutation, which cosegregates with the resistance marker and thus with the T-DNA. The T-DNA is inserted on chromosome 1, BAC F4F7, Accession AC079374, in position 17376. Owing to the insertion at this position, approx. 7 bp downstream of the ORF F4F7.26, it is highly probable that the transcription, or transcript stability, and thus the functionality of this open reading frame is destroyed. This ORF enclodes a putative t-RNA glutamine synthetase and has homology in particular with the Lupinus luteus tRNA glutamine synthetase GI:2995454.

### Example 44

Line 304139 (SEQ ID NO: 104 [nucleic acid] and SEQ ID NO: 105 [protein encoded by the above nucleic acid]) was identified analogously to the abovementioned examples. Line 304139 segregates for an albino-lethal mutation, which cosegregates with the resistance marker and thus with the T-DNA. The T-DNA is inserted on chromosome 5, P1 clone MFB 13, Accession AB010073, in position 49311-49335. Owing to the insertion at this position approx. 25 bp downstream of the ORF MFB13.17, it is highly probable that the transcription, or transcript stability, and thus the functionality of this open reading frame, which encodes an exonuclease-like protein, is destroyed.

### Example 45

Line 304886 (SEQ ID NO: 106 [nucleic acid] and SEQ ID NO: 107 [protein encoded by the above nucleic acid]) was identified analogously to the abovementioned examples. Line 304886 segregates for an albino-lethal mutation, which cosegregates with the resistance marker and thus with the T-DNA. The T-DNA is inserted on chromosome 2, BAC clone F23H14, section 1 of 255, Accession AC006837, in position 84045. Owing to the insertion at this position, the ORF At2g01110 is interrupted and inactivated. This ORF encodes a putative “sec-independent” translocase protein TATC. The sequence is also described in WO 144277.

### Example 46

Line 306053 (SEQ ID NO: 108 [nucleic acid] and SEQ ID NO: 109 [protein encoded by the above nucleic acid]) was identified analogously to the abovementioned examples. Line 306053 segregates for an albino-lethal mutation, which cosegregates with the resistance marker and thus with the T-DNA. The T-DNA is inserted on chromosome 4, BAC clone F28J12, Accession AL021710, in position 74806-74828. Owing to the insertion at this position, the ORF F28J12.180, which encodes a putative protein, is interrupted and inactivated. blastp analyses with standard setting revealed that the derived amino acid sequence has not only pronounced homologies with a variety of hypothetical and putative proteins, but also a high degree of similarity with selenium-binding-protein-like proteins.

1. A method of identifying herbicidally active substances, which comprises influencing the expression or the activity of the gene product of a nucleic acid or of a gene encompassing:
aa) nucleic acid sequence with the sequence shown in SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, SEQ ID NO: 78, SEQ ID NO: 80, SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 98, SEQ ID NO: 100, SEQ ID NO: 102, SEQ ID NO: 106 or SEQ ID NO: 108
or
bb) nucleic acid sequence which, owing to the degeneracy of the genetic code, can be derived by back translation from the amino acid sequences shown in SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 75, SEQ ID NO: 77, SEQ ID NO: 79, SEQ ID NO: 81, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 99, SEQ ID NO: 101, SEQ ID NO: 103, SEQ ID NO: 105, SEQ ID NO: 107 or SEQ ID NO: 109;
cc) nucleic acid sequence which is a derivative or a fragment of the nucleic acid sequences shown in SEQ ID NO: 1, SEQ ID NO: 3, SEQ ID NO: 5, SEQ ID NO: 7 SEQ ID NO: 9, SEQ ID NO: 11, SEQ ID NO: 13, SEQ ID NO: 15, SEQ ID NO: 17, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, SEQ ID NO: 78, SEQ ID NO: 80, SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 98, SEQ ID NO: 100, SEQ ID NO: 102, SEQ ID NO: 106 or SEQ ID NO: 108 and which has at least 60% homology at the nucleic acid level;
dd) nucleic acid sequence which encodes derivatives or fragments of the polypeptides with the amino acid sequences shown in SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 75, SEQ ID NO: 77, SEQ ID NO: 79, SEQ ID NO: 81, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 99, SEQ ID NO: 101, SEQ ID NO: 103, SEQ ID NO: 105, SEQ ID NO: 107 or SEQ ID NO: 109
which have at least 50% homology at the amino acid level;
ee) nucleic acid sequence which encodes a fragment or an epitope of a polypeptide which specifically binds to an antibody, the antibody binding specifically to a polypeptide encoded by the sequence shown in SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, SEQ ID NO: 78, SEQ ID NO: 80, SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 98, SEQ ID NO: 100, SEQ ID NO: 102, SEQ ID NO: 106 or SEQ ID NO: 108;
ff) nucleic acid sequence which encodes a fragment of a nucleic acid shown in aa) and which has m6A-methyltransferase activity, DNA-binding activity or DNA repair activity, for example as in the case of RAD 54, thioredoxin activity, VAV2 activity, fructokinase activity, zinc finger protein activity, LYTB activity, crepopin activity, leucine protein activity, DNAJ activity, CRS1 activity, alanyl-tRNA-synthetase activity, OEP86 activity, FMRF-amide-propeptide isolog activity, 26S proteosome subunit S5B activity, geranylgeranyl-pyrophosphate synthase activity, cecropin family signature, ftsH chloroplast protease activity, AIM1 activity, UDP-glucuronyl transferase activity, FPF1 activity, SHI-like zinc finger protein activity, Crp1 activity, CRS1 activity, translation releasing factor RF-1 activity, farnesyl transferase subunit A activity, ATP-dependent copper transporter RAN1 activity, syntaxin or syntaxin-like-protein activity, inositol-polyphosphate-5′-phosphatase activity, UDP—N-acetylmuramoylalanyl-D-glutamate-2,6-diamino-pimelate ligase activity (murE), β-glucosidase activity, hydroxymethylglutaryl-CoA reductase activity, GDSL-motif-lipase/hydroxylase-like-protein activity, cellulose-synthase-like-protein activity, tRNA-glutamine synthetase activity, exonuclease-like-protein activity, sec-independent translocase protein TATC activity or selenium-binding-protein-like-protein activity; and/or
gg) nucleic acid sequence which encodes derivatives of the polypeptides with the amino acid sequences shown in SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 45, SEQ ID NO: 47, SEQ ID NO: 49, SEQ ID NO: 51, SEQ ID NO: 53, SEQ ID NO: 55, SEQ ID NO: 57, SEQ ID NO: 59, SEQ ID NO: 61, SEQ ID NO: 63, SEQ ID NO: 65, SEQ ID NO: 67, SEQ ID NO: 69, SEQ ID NO: 71, SEQ ID NO: 73, SEQ ID NO: 75, SEQ ID NO: 77, SEQ ID NO: 79, SEQ ID NO: 81, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 99, SEQ ID NO: 101, SEQ ID NO: 103, SEQ ID NO: 105, SEQ ID NO: 107 or SEQ ID NO: 109 which has at least 20% homology at the amino acid level and an equivalent biological activity; or
the expression or activity of an amino acid sequence which is encoded by a nucleic acid sequence of aa) to gg),
and selecting the substances which reduce or block the expression or the activity.

2. A method as claimed in claim 1, wherein the expression or the activity of the nucleic acid or amino acid is reduced or blocked by reducing or blocking the
a) transcription,
b) translation,
c) processing and/or
d) modification
of the nucleic acid sequence or amino acid sequence in claim 1.

3. A method as claimed in claim 1, wherein the substances are identified in a high-throughput screening (HTS).

4. A method as claimed in claim 1, wherein the substances selected are applied to a plant in order to test the herbicidal activity of the substances and those substances which show herbicidal activity are selected.

5. A method as claimed in claim 1, wherein the method is carried out in an organism.

6. A method as claimed in claim 1, wherein the organisms used are bacteria, yeasts, fungi or plants.

7. A method as claimed in claim 1, wherein an organism is used which is a conditional or natural mutant of one of the sequences described in claim 1.

8. A nucleic acid construct comprising a nucleic acid sequence shown in claim 1, wherein the nucleic acid sequence is linked to one or more regulatory signals.

9. A substance identified by a method as claimed in claim 1, the substance having a molecular weight of under 1000 Daltons, a Ki value of under 1 mM and less than three hydroxyl groups on a carbon-atom-containing ring.

10. A substance identified by a method as claimed in claim 1, wherein the substance is a proteinogenic substance or an antisense RNA.

11. A substance as claimed in claim 1, wherein the substance is a proteinogenic substance, and which substance is an antibody against the protein encoded by one of the sequences shown in claim 8.

12. A nucleic acid construct as claimed in claim 8, wherein additional further nucleic acid sequences are present in the nucleic acid construct.

13. A vector comprising a nucleic acid construct as claimed in claim 8.

14. An organism comprising at least one nucleic acid construct as claimed in claim 8 or at least one vector comprising the nucleic acid construct.

15. An organism as claimed in claim 14, which is a plant, a microorganism or an animal.

16. A transgenic plant comprising a functional or nonfunctional nucleic acid construct as claimed in claim 8 or a vector comprising the nucleic acid construct.

17. (canceled)

18. A method of identifying an antagonist of proteins which are encoded by a nucleic acid sequence as set forth in claim 8 by following through the following process steps:
i contacting cells which express the protein, or the protein, with a candidate substance;
ii assaying the biological activity of the protein;
iii comparing the biological activity of the protein with a standard activity in the absence of the candidate substance, a reduced biological activity of the protein indicating that the candidate substance is an antagonist.

19. A method as claimed in claim 18, wherein the antagonist identified in accordance with claim 18, iii), is applied to a plant to test its herbicidal activity, and those antagonists which show a herbicidal activity are selected.

20. A method of controlling undesired vegetation, which comprises allowing a herbicidally active amount of a substance identified by a method as claimed in claim 1 or of an antagonist of a protein encoded by a nucleic acid sequence as set forth in claim 1 identified by
i contacting cells which express the protein, or the protein, with a candidate substance;
ii assaying the biological activity of the protein;
iii comparing the biological activity of the protein with a standard activity in the absence of the candidate substance, a reduced biological activity of the protein indicating that the candidate substance is an antagonist, to act on plants or their environment.

21. (canceled)

22. A method for generating modified gene products encoded by the nucleic acid sequences SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, SEQ ID NO: 78, SEQ ID NO: 80, SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 98, SEQ ID NO: 100, SEQ ID NO: 102, SEQ ID NO: 106 or SEQ ID NO: 108
which comprises the following process steps:
a) expression of the proteins encoded by SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 44, SEQ ID NO: 46, SEQ ID NO: 48, SEQ ID NO: 50, SEQ ID NO: 52, SEQ ID NO: 54, SEQ ID NO: 56, SEQ ID NO: 58, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 74, SEQ ID NO: 76, SEQ ID NO: 78, SEQ ID NO: 80, SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 98, SEQ ID NO: 100, SEQ ID NO: 102, SEQ ID NO: 106 or SEQ ID NO: 108 in a heterologous system or in a cell-free system
b) randomized or directed mutagenesis of the protein by modification of the nucleic acid,
c) measuring the interaction of the modified gene product with the herbicide or the biological activity of the modified gene product in the presence of the herbicide,
d) identification of derivatives of the protein which exhibit a lesser degree of interaction or whose activity is less affected,
e) assaying the biological activity of the protein following application of the herbicide,
f) selection of the nucleic acid sequences which, or whose gene products, show a modified biological activity with regard to the herbicide.

23. A method as claimed in claim 22, wherein the sequences selected in accordance with claim 22 f) are introduced into an organism.

24. A method for generating transgenic plants which are resistant to substances found by a method as claimed in claim 1 or a method of identifiying an antagonist of proteins which are encoded by a nucleic acid sequence as set forth in claim 1 by following through the following process steps:
i contacting cells which express the protein, or the protein, with a candidate substance;
ii assaying the biological activity of the protein;
iii comparing the biological activity of the protein with a standard activity in the absence of the candidate substance, a reduced biological activity of the protein indicating that the candidate substance is an antagonist,
which comprises overexpressing, in these plants, nucleic acids with the sequences described in claim 1.

25. An organism generated by a method as claimed in claim 22.

26. A composition comprising a herbicidally active amount of at least one substance identified by a method as claimed in claim 1 or of an antagonist identified by a method of identifying an antagonist of proteins which are encoded by a nucleic acid sequence as set forth in claim 1 by following through the following process steps:
i contacting cells which express the protein, or the protein, with a candidate substance;
ii assaying the biological activity of the protein;
iii comparing the biological activity of the protein with a standard activity in the absence of the candidate substance, a reduced biological activity of the protein indicating that the candidate substance is an antagonist and at least one inert liquid and/or solid carrier and, optionally, at least one surfactant.

27. A composition comprising a growth-regulating amount of at least one substance identified by a method as claimed in claim 1 or of an antagonist identified by a method of identifying an antagonist of proteins which are encoded by a nucleic acid sequence as set forth in claim 1 by following through the following process steps:
i contacting cells which express the protein, or the protein, with a candidate substance;
ii assaying the biological activity of the protein;
iii comparing the biological activity of the protein with a standard activity in the absence of the candidate substance, a reduced biological activity of the protein indicating that the candidate substance is an antagonist and at least one inert liquid and/or solid carrier and, optionally, at least one surfactant.

28. A composition comprising the substance as claimed in claim 9.

29. A kit encompassing the nucleic acid construct as claimed in claim 8.

30. An organism generated by a method as claimed in claim 24.

31. A composition comprising the substance as claimed in claim 18.

32. A substance as claimed in claim 1, where in the substance is an antisense RNA comprising a nucleic acid molecule as shown in claim 1 or a fragment thereof.
